(19) United States
(12) Patent Application Publication
(10) Pub. No.: US 2019/0151416 A1
(43) Pub. Date: May 23, 2019

(54) FIBROBLAST GROWTH FACTOR (FGF) 1 PROTEINS WITH GLUCOSE LOWERING ABILITY AND REDUCED MITOGENICITY

(71) Applicant: Salk Institute for Biological Studies, La Jolla, CA (US)

(72) Inventors: Ronald M. Evans, La Jolla, CA (US); Michael Downes, La Jolla, CA (US); Annette Atkins, La Jolla, CA (US); Ruth T. Yu, La Jolla, CA (US); Sihao Liu, La Jolla, CA (US)

(73) Assignee: Salk Institute for Biological Studies, La Jolla, CA (US)

(21) Appl. No.: 16/252,292

(22) Filed: Jan. 18, 2019

Related U.S. Application Data
(63) Continuation of application No. PCT/US2017/044678, filed on Jul. 31, 2017.
(60) Provisional application No. 62/369,460, filed on Aug. 1, 2016.

(51) Int. Cl. A61K 38/18; C07K 14/50; A61P 3/10; C12N 15/62; A61P 1/16; A61K 31/4439

(52) U.S. Cl. CPC A61K 38/1825; C07K 14/501; A61K 31/4439; C12N 15/625; A61P 1/16; A61P 3/10

(57) ABSTRACT

The present disclosure provides FGF1 mutant proteins, which include an N-terminal deletion, point mutation(s), or combinations thereof. In some examples, the mutant FGF1 proteins have reduced mitogenic activity. Also provided are nucleic acid molecules that encode such proteins, and vectors and cells that include such nucleic acids. The disclosed FGF1 mutants can reduce blood glucose in a mammal, and in some examples are used to treat a metabolic disorder.

### CROSS-REFERENCE TO RELATED APPLICATIONS

This application is a continuation of International Application No. PCT/US2017/044678, filed Jul. 31, 2017, which claims priority to U.S. Provisional Application No. 62/369,460 filed Aug. 1, 2016, both herein incorporated by reference.

### FIELD

This application provides mutated FGF1 proteins, nucleic acids encoding such proteins, and methods of their use, for example to reduce blood glucose and/or to treat a metabolic disease. In some examples, these mutants have significantly reduced mitogenicity.

### BACKGROUND

Type 2 diabetes and obesity are leading causes of mortality and are associated with the Western lifestyle, which is characterized by excessive nutritional intake and lack of exercise. A central player in the pathophysiology of these diseases is the nuclear hormone receptor (NHR) PPARγ, a lipid sensor and master regulator of adipogenesis. PPARγ is also the molecular target for the thiazolidinedione (TZD)-class of insulin sensitizers, which command a large share of the current oral anti-diabetic drug market. However, there are numerous side effects associated with the use of TZDs such as weight gain, liver toxicity, upper respiratory tract infection, headache, back pain, hyperglycemia, fatigue, sinusitis, diarrhea, hypoglycemia, mild to moderate edema, and anemia. Thus, the identification of new insulin sensitizers is needed.

### SUMMARY

It was previously observed that some N-terminally truncated FGF1 mutants failed to lower glucose because their protein stability was too compromised. For example, removal of the N-terminal 9 amino acids (FGF1 (10-140 aa); (aa 10-140 of SEQ ID NO: 5)) lowered glucose similar to full length mature FGF1 (1-140 aa) (SEQ ID NO: 5), but removal of the N-terminal 11 amino acids FGF1 (12-140aa) (aa 13-140 of SEQ ID NO: 5) reduced glucose lowering activity, and removal of the N-terminal 13 amino acids (FGF1 (14-140 aa) (aa 14-140 of SEQ ID NO: 5)) resulted in a protein with no significant effect on glucose lowering in vivo. It is shown herein that if mutations are introduced into N-terminally truncated proteins, which increase the thermodynamic stability, glucose lowering activity is restored. For example, introducing the combination of mutations H²¹Y, L⁴⁴F, H¹⁰²Y, and F¹⁰⁸Y into an FGF1 containing a 13 aa N-terminal deletion (SEQ ID NO: 17) rescues the glucose lowering activity, but the combination Q⁴⁰P, S⁴⁷I, and H⁹³G (SEQ ID NO: 16) did not.

Based on these observations, mutant FGF1 proteins (and encoding nucleic acid molecules) are provided. Such mutants can include an N-terminal truncation, one or more point mutation(s) (such as those that increase stability of the protein), or combinations thereof. Methods of using the mutant FGF1 proteins/nucleic acid molecules for reducing blood glucose in a mammal, for example to treat a metabolic disease, are disclosed. In some examples, the FGF1 mutants are mutated to reduce the mitogenic activity, alter heparan sulfate and/or heparin binding, and/or increase the thermostability of the FGF1 mutant protein (e.g., relative to a native FGF1 protein). Such FGF1 mutants can be used alone or in combination with other agents, such as other glucose reducing agents, such as thiazolidinedione. In some examples, use of the disclosed mutant FGF1 proteins result in one or more of: reduction in triglycerides, decrease in insulin resistance, reduction of hyperinsulinemia, increase in glucose tolerance, reduction of food intake, or reduction of hyperglycemia in a mammal.

Provided herein are mutated FGF1 proteins containing an N-terminal truncation, one or more point mutation(s) (such as amino acid substitutions, deletions, additions, or combinations thereof), or combinations of N-terminal deletions and point mutation(s). In some examples, such mutated FGF1 proteins have reduced mitogenicity relative to mature FGF1 (e.g., SEQ ID NO: 5), such as a reduction of at least 20%, at least 50%, at least 75% or at least 90%. In some examples, mutated FGF1 proteins have increased thermostability relative to mature FGF1 (e.g., SEQ ID NO: 5), such as an increase of at least 20%, at least 50%, at least 75%, at least 90%, at least 100%, or at least 200%. In some examples, the mutant FGF1 protein can include for example deletion of at least 5, at least 6, at least 10, at least 11, at least 12, at least 13, at least 14, at least 15, at least 16, at least 17, at least 18, at least 19, or at least 20 consecutive N-terminal amino acids. In some examples, the mutant FGF1 protein includes point mutations, such as one containing at least 1, at least 2, at least 3, at least 4, at least 5, at least 6, at least 7, at least 8, at least 9, or at least 10 additional amino acid substitutions (such as 1, 2, 3, 4, 5, 6, 7, 8, 9, 10, 11, 12, 13, 14, 15, 16, 17, 18, or 19 substitutions), such as one or more of those shown in Table 1. In some examples, the mutant FGF1 protein includes both an N-terminal truncation and one or more additional point mutations. In some examples, the mutant FGF1 protein includes at least 90, at least 100, or at least 110 consecutive amino acids from amino acids 5-141 of FGF1 (e.g., of SEQ ID NO: 2, 4 or 5), (which in some examples can include 1-20 point mutations, such as substitutions, deletions, and/or additions).

Also provided are isolated nucleic acid molecules encoding the disclosed mutant FGF1 proteins. Vectors and cells that include such nucleic acid molecules are also provided.

Methods of using the disclosed mutant FGF1 proteins (or nucleic acid molecules encoding such) are provided. In some examples the methods include administering a therapeutically effective amount of one or more disclosed mutant FGF1 proteins (or nucleic acid molecules encoding such) to reduce blood glucose in a mammal, such as a decrease of at least 5%, at least 10%, at least 25%, at least 50%, or at least 75%. In some examples the methods include administering a therapeutically effective amount of a disclosed mutant FGF1 protein (or nucleic acid molecules encoding such) to treat a metabolic disease in a mammal. Exemplary metabolic diseases that can be treated with the disclosed methods include, but are not limited to: diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension). In some examples, one or more of these diseases are treated simultaneously with the disclosed FGF1 mutants. Also provided are methods of reducing fed and fasting blood glucose, improving insulin sensitivity and glucose tolerance, reducing systemic chronic inflammation, ameliorating hepatic steatosis in a mammal, reducing food intake, or combinations thereof, by administering a therapeutically effective amount of a disclosed mutant FGF1 protein (or nucleic acid molecules encoding such).

The foregoing and other objects and features of the disclosure will become more apparent from the following detailed description, which proceeds with reference to the accompanying figures.

### BRIEF DESCRIPTION OF THE DRAWINGS

FIGS. 1A-1O provide several exemplary FGF1 mutant proteins (A) mature FGF1 with four point mutations (K12V, A66C, N95V, C117V) (SEQ ID NO: 10); (B) mature form of FGF1 with four point mutations (Y55W, E87H, S116R, C117V) (SEQ ID NO: 11); (C) mature form of FGF1 with five point mutations (K12V, Y55W, N95V, S116R, C117V) (SEQ ID NO: 12); (D) N-terminally truncated form of FGF1 with six point mutations (K12V, L44F, C83T, N95V, C117V, F132W), wherein some of the N-terminus is replaced with an engineered N-terminal sequence (NF21) (SEQ ID NO: 13); (E) N-terminally truncated form of FGF1 with seven point mutations (K12V, H21Y, L44F, N95V, H102Y, F108Y, C117V), wherein some of the N-terminus is replaced with NF21 (SEQ ID NO: 14); (F) N-terminally truncated form of FGF1 with three point mutations (K12V, E87V, C117V), wherein some of the N-terminus is replaced with NF21 (SEQ ID NO: 15); (G) N-terminally truncated FGF1 (14-140 αα) with four point mutations (Q40P, S47I, H93G, and N95V) (SEQ ID NO: 16), (H) N-terminally truncated FGF1 (FGF1^{ΔNT}(14-140 αα) with five point mutations (H21Y, L44F, N95V, H102Y, F108Y) (SEQ ID NO: 17), (I) N-terminally truncated FGF1 (14-140 αα) with six point mutations (H21Y, L44F, N95V, H102Y, F108Y and C117V) (SEQ ID NO: 18), (J) N-terminally truncated FGF1 (14-140 αα) with five point mutations (L44F, C83T, N95V, F132W and C117V) (SEQ ID NO: 19), (K) N-terminally truncated form of FGF1 with six point mutations (H21Y, L44F, N95V, H102Y, F108Y and C117V), wherein some of the N-terminus is replaced with NF21 (SEQ ID NO: 20); (L) N-terminally truncated form of FGF1 with five point mutations (H21Y, L44F, N95V, H102Y, and F108Y), wherein some of the N-terminus is replaced with NF21 (SEQ ID NO: 21); (M) N-terminally truncated FGF1 (12-140 αα) with five point mutations (K12V, Q40P, S47I, H93G, and N95V) (SEQ ID NO: 22), (N) N-terminally truncated FGF1 (12-140 αα) with six point mutations (K12V, H21Y, L44F, N95V, H102Y, and F108Y), SEQ ID NO: 23) and (O) N-terminally truncated FGF1 (12-140 αα) with two point mutations (K12V and N95V) (SEQ ID NO: 24).

FIG. 2 shows an alignment between different mammalian wild-type FGF1 sequences (human (SEQ ID NO: 2), gorilla (SEQ ID NO: 6), chimpanzee (SEQ ID NO: 7), canine (SEQ ID NO: 8), feline (SEQ ID NO: 8), and mouse (SEQ ID NO: 4)). Such an alignment can be routinely generated in the art, and can be used to make the mutations provided herein to any FGF1 sequence of interest.

FIG. 3A-3B are a series of graphs showing the (A) in vivo blood glucose lowering effects and (B) in vitro mitogenicity of human FGF1 (SEQ ID NO: 2) and the FGF1 mutant Salk_075 (SEQ ID NO: 10).

FIG. 4A-4C are a series of graphs showing the (A) in vivo blood glucose lowering effects and (B, C) in vitro mitogenicity of human FGF1 (SEQ ID NO: 2), and the FGF1 mutants Salk_076 (SEQ ID NO: 11) and Salk_077 (SEQ ID NO: 12).

FIG. 5A-5C are a series of graphs showing the (A) in vivo blood glucose lowering effects and (B, C) in vitro mitogenicity of human FGF1 (SEQ ID NO: 2) and the FGF1 mutants Salk_102-1 (SEQ ID NO: 16) and Salk_102-2 (SEQ ID NO: 17).

FIG. 6 shows the amino acid sequence of FGF1 (SEQ ID NO: 5) and FGF2 (SEQ ID NO: 26), and the location of the 12 beta strands. Mutations can be made to the loop between beta strands 9 and 10 to reduce mitogenicity without affecting receptor binding. The equivalent mutations in FGF1 are shown (S99A, K101E, H102A, and W107A) (SEQ ID NO: 25).

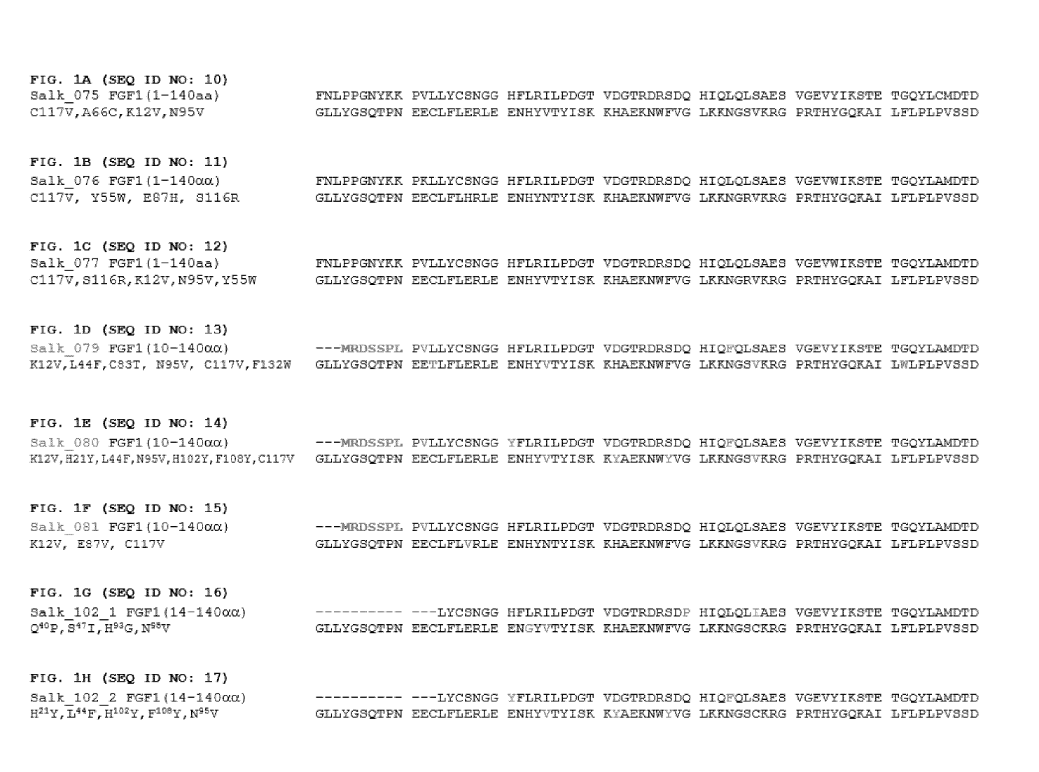

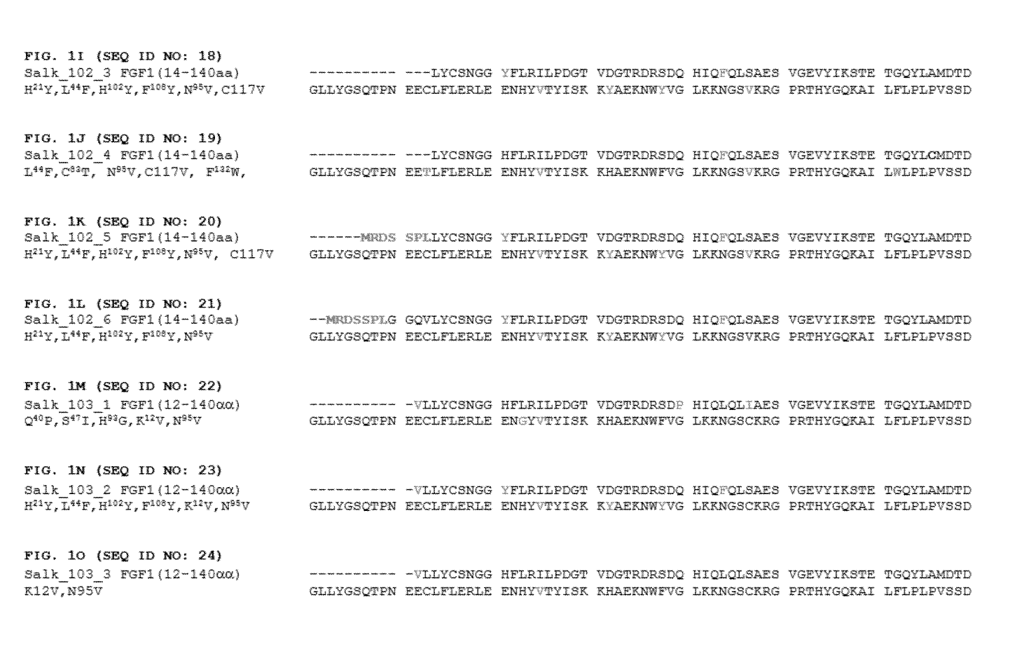

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing provided herewith (sequence listing.txt, created on Jan. 12, 2019, 40 kb) is part of the specification, and is incorporated by reference.

SEQ ID NOS: 1 and 2 provide an exemplary human FGF1 nucleic acid and protein sequences, respectively. Source: GenBank Accession Nos: BC032697.1 and AAH32697.1. Heparan binding residues are amino acids 127-129 and 133-134.

SEQ ID NOS: 3 and 4 provide an exemplary mouse FGF1 nucleic acid and protein sequences, respectively. Source: GenBank Accession Nos: BC037601.1 and AAH37601.1.

SEQ ID NO: 5 provides an exemplary mature form of FGF1 (140 αα, sometimes referred to in the art as FGF1 15-154).

SEQ ID NO: 6 provides an exemplary gorilla FGF1 protein sequence.

SEQ ID NO: 7 provides an exemplary chimpanzee FGF1 protein sequence.

SEQ ID NO: 8 provides an exemplary dog FGF1 protein sequence.

SEQ ID NO: 9 provides an exemplary cat FGF1 protein sequence.

SEQ ID NO: 10 (Salk_075) provides an exemplary mature form of FGF1 with four point mutations (K12V, A66C, N95V, C117V) wherein numbering refers to SEQ ID NO: 5).

SEQ ID NO: 11 (Salk_076) provides an exemplary mature form of FGF1 with four point mutations (Y55W, E87H, S116R, C117V, wherein numbering refers to SEQ ID NO: 5).

SEQ ID NO: 12 (Salk_077) provides an exemplary mature form of FGF1 with five point mutations (K12V, Y55W, N95V, S116R, C117V, wherein numbering refers to SEQ ID NO: 5). SEQ ID NO: 13 (Salk_079) provides an exemplary N-terminally truncated form of FGF1 with six point mutations (K12V, L44F, C83T, N95V, C117V, F132W, wherein numbering refers to SEQ ID NO: 5), wherein some of the N-terminus is replaced with NF21.

SEQ ID NO: 14 (Salk_080) provides an exemplary N-terminally truncated form of FGF1 with seven point mutations (K12V, H21Y, L44F, N95V, H102Y, F108Y, C117V, wherein numbering refers to SEQ ID NO: 5), wherein some of the N-terminus is replaced with NF21.

SEQ ID NO: 15 (Salk_081) provides an exemplary N-terminally truncated form of FGF1 with three point mutations (K12V, E87V, C117V, wherein numbering refers to SEQ ID NO: 5), wherein some of the N-terminus is replaced with NF21.

SEQ ID NO: 16 (Salk_102_1) provides an exemplary N-terminally truncated form of FGF1 with four point mutations (Q40P, S47I, H93G, and N95V, wherein numbering refers to SEQ ID NO: 5).

SEQ ID NO: 17 (Salk_102_2) provides an exemplary N-terminally truncated form of FGF1 with five point mutations (H21Y, L44F, N95V, H102Y, and F108Y, wherein numbering refers to SEQ ID NO: 5).

SEQ ID NO: 18 (Salk_102_3) provides an exemplary N-terminally truncated form of FGF1 with six point mutations (H21Y, L44F, N95V, H102Y, F108Y, and C117V, wherein numbering refers to SEQ ID NO: 5).

SEQ ID NO: 19 (Salk_102_4) provides an exemplary N-terminally truncated form of FGF1 with five point mutations (L44F, C83T, N95V, F132W, and C117V, wherein numbering refers to SEQ ID NO: 5).

SEQ ID NO: 20 (Salk_102_5) provides an exemplary N-terminally truncated form of FGF1 with five point mutations (H21Y, L44F, N95V, H102Y, F108Y, and C117V wherein numbering refers to SEQ ID NO: 5), wherein some of the N-terminus is replaced with NF21.

SEQ ID NO: 21 (Salk_102_6) provides an exemplary N-terminally truncated form of FGF1 with five point mutations (H21Y, L44F, N95V, H102Y, and F108Y, wherein numbering refers to SEQ ID NO: 5), wherein some of the N-terminus is replaced with NF21.

SEQ ID NO: 22 (Salk_103_1) provides an exemplary N-terminally truncated form of FGF1 with five point mutations (K12V, Q40P, S47I, H93G, and N95V, wherein numbering refers to SEQ ID NO: 5).

SEQ ID NO: 23 (Salk_103_2) provides an exemplary N-terminally truncated form of FGF1 with five point mutations (K12V, H21Y, L44F, N95V, H102Y, and F108Y, wherein numbering refers to SEQ ID NO: 5).

SEQ ID NO: 24 (Salk_103_3) provides an exemplary N-terminally truncated form of FGF1 with two point mutations (K12 and N95V, wherein numbering refers to SEQ ID NO: 5).

SEQ ID NO: 25 provides an exemplary mature form of FGF1 with four point mutations (S99A, K101E, H102A, and W107, wherein numbering refers to SEQ ID NO: 5). One, two, three of all four of these point mutations can be made to an FGF1 sequence (such as a mutant FGF1 protein provided herein) to reduce its mitogenicity.

SEQ ID NO: 26 provides an exemplary portion of a human FGF2 protein sequence.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a protein” includes single or plural proteins and is considered equivalent to the phrase “comprising at least one protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Aug. 1, 2016. All references and GenBank® Accession numbers cited herein are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject an agent, such as a mutated FGF1 protein or nucleic acid molecule disclosed herein, by any effective route. Exemplary routes of administration include, but are not limited to, oral, injection (such as subcutaneous, intramuscular, intradermal, intraperitoneal, intravenous, intrathecal, and intratumoral), sublingual, rectal, transdermal, intranasal, vaginal and inhalation routes.

C-terminal portion: A region of a protein sequence that includes a contiguous stretch of amino acids that begins at or near the C-terminal residue of the protein. A C-terminal portion of the protein can be defined by a contiguous stretch of amino acids (e.g., a number of amino acid residues).

Diabetes mellitus: A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 2 diabetes results from insulin resistance, a condition in which cells fail to use insulin properly, sometimes combined with an absolute insulin deficiency. This form is also called “non-insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and in some examples diagnosed by demonstrating any one of:
a. Fasting plasma glucose level≥7.0 mmol/l (126 mg/dl);
b. Plasma glucose≥11.1 mmol/l (200 mg/dL) two hours after a 75 g oral glucose load as in a glucose tolerance test;
c. Symptoms of hyperglycemia and casual plasma glucose≥11.1 mmol/l (200 mg/dl);
d. Glycated hemoglobin (Hb A1C)≥6.5%

Effective amount or therapeutically effective amount: The amount of agent, such as a mutated FGF1 protein (or nucleic acid encoding such) disclosed herein, that is an amount sufficient to prevent, treat (including prophylaxis), reduce, and/or ameliorate the symptoms and/or underlying causes of any of a disorder or disease. In one embodiment, an “effective amount” is sufficient to reduce or eliminate a symptom of a disease, such as a diabetes (such as type II diabetes), for example by lowering blood glucose.

Fibroblast Growth Factor 1 (FGF1): e.g., OMIM 13220. Includes FGF1 nucleic acid molecules and proteins. FGF1 is a protein that binds to the FGF receptor and is also known as the acidic FGF. FGF1 sequences are publically available, for example from GenBank® sequence database (e.g., Accession Nos. NP_00791 and NP_034327 provide exemplary FGF1 protein sequences, while Accession Nos. NM_000800 and NM_010197 provide exemplary FGF1 nucleic acid sequences). One of ordinary skill in the art can identify additional FGF1 nucleic acid and protein sequences, including FGF1 variants.

Specific examples of native FGF1 sequences are provided in SEQ ID NOS: 1-5. A native FGF1 sequence is one that does not include a mutation that alters the normal activity of the protein (e.g., activity of SEQ ID NOS: 2, 4 or 5). A mature FGF1 refers to an FGF1 peptide or protein product and/or sequence following any post-translational modifications. A mutated FGF1 is a variant of FGF1 with different or altered biological activity, such as reduced mitogenicity (e.g., a variant of any of SEQ ID NOS: 1-5, such as one having at least 90%, at least 95%, at least 96%, at least 97%, at least 98% or at least 99% sequence identity to any of SEQ ID NOS: 1-5, but is not a native/wild-type sequence). In one example, such a variant includes an N-terminal truncation and/or one or more additional point mutations (such as one or more of those shown in Table 1), such as changes that decrease mitogenicity of FGF1, alter the heparin binding affinity of FGF1, and/or the thermostability of FGF1. Specific exemplary FGF1 mutant proteins are shown in SEQ ID NOS: 10-25.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. Thus, host cells can be transgenic, in that they include nucleic acid molecules that have been introduced into the cell, such as a nucleic acid molecule encoding a mutant FGF1 protein disclosed herein.

Isolated: An “isolated” biological component (such as a mutated FGF1 protein or nucleic acid molecule) has been substantially separated, produced apart from, or purified away from other biological components in the cell of the organism in which the component naturally occurs, such as other chromosomal and extrachromosomal DNA and RNA, and proteins. Nucleic acid molecules and proteins which have been “isolated” thus include nucleic acids and proteins purified by standard purification methods. The term also embraces nucleic acid molecules and proteins prepared by recombinant expression in a host cell as well as chemically synthesized nucleic acids. A purified or isolated cell, protein, or nucleic acid molecule can be at least 70%, at least 80%, at least 90%, at least 95%, at least 96%, at least 97%, at least 98%, or at least 99% pure.

Mammal: This term includes both human and non-human mammals. Similarly, the term “subject” includes both human and veterinary subjects (such as cats, dogs, cows, and pigs) and rodents (such as mice and rats).

Metabolic disorder/disease: A disease or disorder that results from the disruption of the normal mammalian process of metabolism. For example, a metabolic disorder/disease includes metabolic syndrome.

Other examples include, but are not limited to, (1) glucose utilization disorders and the sequelae associated therewith, including diabetes mellitus (Type I and Type-2), gestational diabetes, hyperglycemia, insulin resistance, abnormal glucose metabolism, “pre-diabetes” (Impaired Fasting Glucose (IFG) or Impaired Glucose Tolerance (IGT)), and other physiological disorders associated with, or that result from, the hyperglycemic condition, including, for example, histopathological changes such as pancreatic β-cell destruction; (2) dyslipidemias and their sequelae such as, for example, atherosclerosis, coronary artery disease, cerebrovascular disorders and the like; (3) other conditions which may be associated with the metabolic syndrome, such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thrombosis, hypercoagulable and prothrombotic states (arterial and venous), hypertension, cardiovascular disease, stroke and heart failure; (4) disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; (5) disorders of cell cycle or cell differentiation processes such as adipose cell tumors, lipomatous carcinomas including, for example, liposarcomas, solid tumors, and neoplasms; (6) neurodegenerative diseases and/or demyelinating disorders of the central and peripheral nervous systems and/or neurological diseases involving neuroinflammatory processes and/or other peripheral neuropathies, including Alzheimer's disease, multiple sclerosis, Parkinson's disease, progressive multifocal leukoencephalopathy, and Guillain-Barre syndrome; (7) skin and dermatological disorders and/or disorders of wound healing processes, including erythemato-squamous dermatoses; and (8) other disorders such as syndrome X, osteoarthritis, and acute respiratory distress syndrome. Other examples are provided in WO 2014/085365 (herein incorporated by reference).

In specific examples, the metabolic disease includes one or more of (such as at least 2 or at least 3 of): diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension).

N-terminal portion: A region of a protein sequence that includes a contiguous stretch of amino acids that begins at or near the N-terminal residue of the protein. An N-terminal portion of the protein can be defined by a contiguous stretch of amino acids (e.g., a number of amino acid residues).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a mutated FGF1 coding sequence). Generally, operably linked DNA sequences are contiguous and, where necessary to join two protein coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the disclosed mutated FGF1 proteins (or nucleic acid molecules encoding such) herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid molecule is one that has a sequence that is not naturally occurring (e.g., a mutated FGF1 protein) or has a sequence that is made by an artificial combination of two otherwise separated segments of sequence. This artificial combination can be accomplished by routine methods, such as chemical synthesis or by the artificial manipulation of isolated segments of nucleic acids, such as by genetic engineering techniques. Similarly, a recombinant protein is one encoded for by a recombinant nucleic acid molecule. Similarly, a recombinant or transgenic cell is one that contains a recombinant nucleic acid molecule and expresses a recombinant protein.

Sequence identity of amino acid sequences: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn, and tblastx. A description of how to determine sequence identity using this program is available on the NCBI website on the internet.

Variants of the mutated FGF1 proteins and coding sequences disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, a mutant FGF1 protein provided herein, can share at least 80%, at least 85%, at least 90%, at least 91%, at least 92%, at least 95%, at least 96%, at least 97%, at least 98% or at least 99% sequence identity to any one of SEQ ID NOS: 10-25, but is not SEQ ID NOS: 2, 4, or 5 (which, in some examples, has one or more, such as 1, 2, 3, 4, 5, 6, 7, 8, 9, or 10 of the mutations shown in Table 1). In addition, exemplary mutated FGF1 proteins have at least 80%, at least 85%, at least 90%, at least 92%, at least 95%, at least 96%, at least 97%, at least 98% or at least 99% sequence identity to any one of SEQ ID NOS: 10-25, and retain the ability to reduce blood glucose levels in vivo.

Subject: Any mammal, such as humans, non-human primates, pigs, sheep, cows, dogs, cats, rodents and the like which is to be the recipient of the particular treatment, such as treatment with a mutated FGF1 protein (or corresponding nucleic acid molecule) provided herein. In two non-limiting examples, a subject is a human subject or a murine subject. In some examples, the subject has one or more metabolic diseases, such as diabetes (e.g., type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular disease (e.g., hypertension), or combinations thereof. In some examples, the subject has elevated blood glucose.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed., Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Transgene: An exogenous gene supplied by a vector. In one example, a transgene includes a mutated FGF1 coding sequence.

Vector: A nucleic acid molecule as introduced into a host cell, thereby producing a transformed host cell. A vector may include nucleic acid sequences that permit it to replicate in the host cell, such as an origin of replication. A vector may also include one or more mutated FGF1 coding sequences and/or selectable marker genes and other genetic elements known in the art. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating, or the like.

### Overview

Provided herein are mutated FGF1 proteins, which can include an N-terminal deletion, one or more additional point mutations (such as amino acid substitutions, deletions, additions, or combinations thereof), or combinations of an N-terminal deletion and additional one or more point mutations.

Also provided are methods of using the disclosed FGF1 mutant proteins (or their nucleic acid coding sequences) to lower glucose, for example to treat one or more metabolic diseases, or combinations thereof. Exemplary metabolic diseases that can be treated with the disclosed methods include, but are not limited to: type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular diseases (e.g., hypertension), latent autoimmune diabetes (LAD), or maturity onset diabetes of the young (MODY).

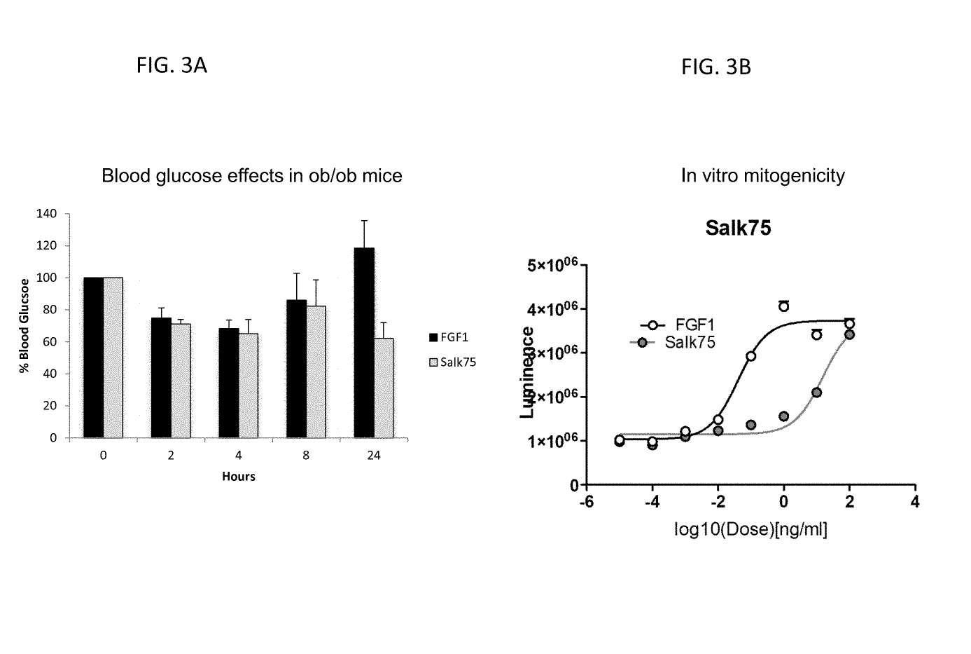

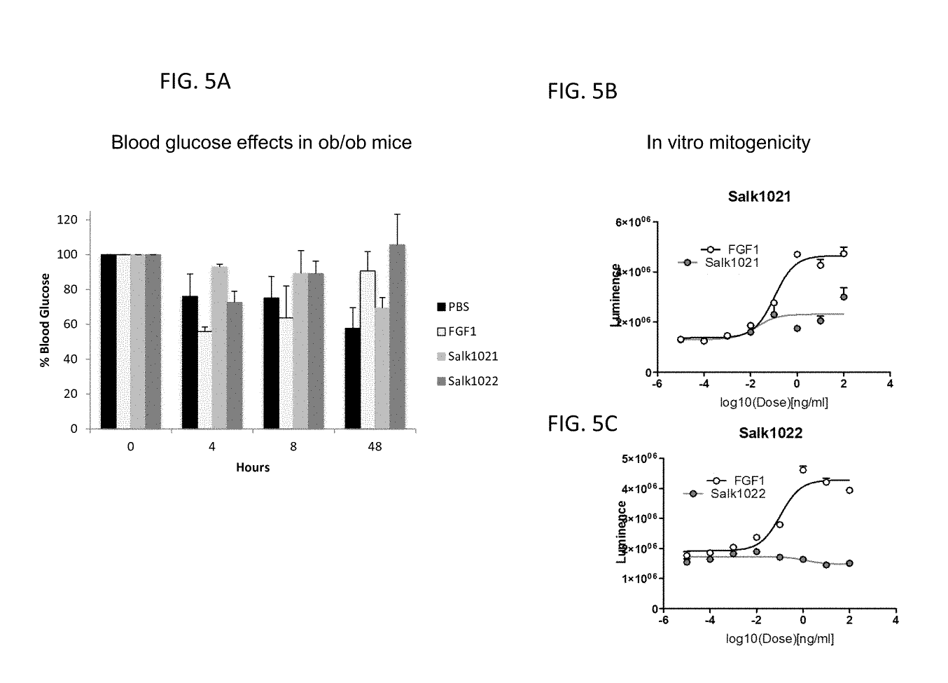

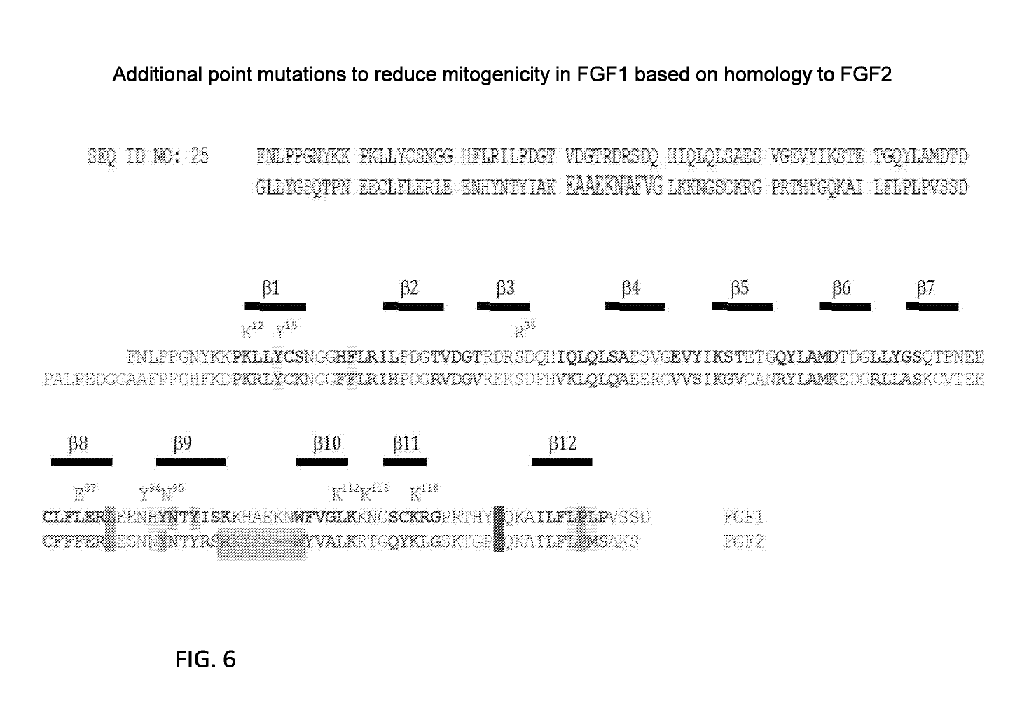

In some examples, an FGF1 mutant protein includes mutations that reduce its mitogenicity (e.g., relative to the mature wild-type FGF1, e.g., SEQ ID NO: 5), such as a reduction of at least 20%, at least 50%, at least 75%, or at least 90%. In some examples, the FGF1 mutant protein has an EC₅₀ for mitogenicity that is shifted by several orders of magnitude relative to the mature wild-type FGF1 (e.g., SEQ ID NO: 5) (for example see FIGS. 3B, 4C, 5B) (such as an EC₅₀ increase of 1 log, 2 logs, or 3 logs), or even no detectable mitogenicity (see FIG. 5C). Methods of measuring mitogenicity are known in the art and are provided herein.

In some examples, an FGF1 mutant protein includes mutations that increase its blood glucose lowering ability relative to the mature wild-type FGF1 (e.g., SEQ ID NO: 5), such as an increase of at least 10%, at least 20%, at least 50%, at least 75%, or at least 90%. In some examples, the FGF1 mutant protein has a similar glucose lowering to mature wild-type FGF1 (e.g., SEQ ID NO: 5). Methods of measuring blood glucose are known in the art and are provided herein.

In some examples, the mutant FGF1 protein includes a truncated version of the mature protein (e.g., SEQ ID NO: 5), which can include for example deletion of at least 5, at least 6, at least 9, at least 10, at least 11, at least 12, at least 13, at least 14, at least 15, or at least 20 consecutive N-terminal amino acids, such as the N-terminal 5 to 10, 5 to 13, 5, 6, 7, 8, 9, 10, 11, 12, 13, 14, 15, 16, 17, 18, 19 or 20 amino acids of mature FGF1. In some examples, such an N-terminally deleted FGF1 protein has reduced mitogenic activity as compared to wild-type mature FGF1 protein. Specific examples of N-terminally deleted FGF1 proteins are shown in SEQ ID NOS: 13-24. In some examples, the N-terminally deleted amino acids are replaced with the engineered sequence MRDSSPL (referred to herein as NF21), for example as shown in SEQ ID NOS: 13-15 and 20-21. Thus, any of SEQ ID NOS: 13-24 can be modified to include one or more of the point mutations shown in Table 1.

In some examples, a mutated FGF1 includes one or more mutations that increase the thermostability (e.g., relative to mature or truncated FGF1, e.g., SEQ ID NO: 5), such as an increase of at least 20%, at least 50%, at least 75% or at least 90% compared to native FGF1. Exemplary mutations that can be used to increase the thermostability include, but are not limited to, (a) one or more of C117V, A66C, K12V, and N95V, (b) one or more of C117V, Y55W, E87H, and S116R, (c) one or more of C117V, S116R, K12V, N95V, and Y55W, (d) one or more of K12V, L44F, C83T, N95V, C117V, and F132W, (e) one or more of K12V, H21Y, L44F, N95V, H102Y, F108Y, and C117V (f) one or more of K12V, E87V, and C117V, (g) one or more of Q40P, S47I, H93G, and N95V, (h) one or more of H21Y, L44F, H102Y, F108Y, and N95V, (i) one or more of H21Y, L44F, H102Y, F108Y, N95V and C117V, (j) one or more of L44F, C83T, N95V, C117V, and F132W, (k) one or more of Q40P, S47I, H93G, K12V, and N95V, (k) one or more of H21Y, L44F, H102Y, F108Y, K12V, and N95V, or (k) one or more of K12V and N95V, wherein the numbering refers to SEQ ID NO: 5. For example, a mutated FGF1 can be mutated to increase the thermostability of the protein relative to an FGF1 protein without the modification. Methods of measuring thermostability are known in the art. In one example, the method provided in Xia et al., PloS One. 7:e48210, 2012 is used.

In some examples, the mutant FGF1 protein includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 amino acid substitutions, such as 1-20, 1-10, 4-8, 5-25, 1, 2, 3, 4, 5, 6, 7, 8, 9, 10, 11, 12, 13, 14, 15, 16, 17, 18, 19, 20, 21, 22, 23, 24, or 25 amino acid substitutions (such as those shown in Table 1). In some examples, the mutant FGF1 protein further includes deletion of one or more amino acids, such as deletion of 1-10, 4-8, 5-10, 1, 2, 3, 4, 5, 6, 7, 8, 9, 10, 11, 12, 13, 14, 15, 16, 17, 18, 19, or 20 amino acid deletions. In some examples, the mutant FGF1 protein includes a combination of amino acid substitutions and deletions, such as at least 1 substitution and at least 1 deletion, such as 1 to 10 substitutions with 1 to 10 deletions.

Exemplary mutations that can be made to a mutant FGF1 protein are shown in Table 1 below, with amino acids referenced to either SEQ ID NOS: 2 or 5. One skilled in the art will recognize that these mutations can be used singly, or in any combination (such as 1-19, 1-10, 4-8, 2-7, 5-25, 1, 2, 3, 4, 5, 6, 7, 8, 9, 10, 11, 12, 13, 14, 15, 16, 17, 18, or 19 of these amino acid substitutions and/or deletions).

TABLE 1
Exemplary FGF1 mutations

| Location of Point | | Location of Point |
|---|---|---|
| Mutation Position | | Mutation Position |
| in SEQ ID NO: 2 | Mutation Citation | in SEQ ID NO: 5 |
| K27 | K12V | K12 |
| H36 | H21Y | H21 |
| Q55 | Q40P | Q40 |
| L59 | L44F | L44 |
| S62 | S47A, S47V, S47I | S47 |
| Y70 | Y55F, Y55V, Y55S, Y55A, Y55W | Y55 |
| A81 | A66C | A66 |
| C98 | C83T, C83S, C83A, C83V | C83 |
| E102 | E87Q, E87D, E87V, E87A, | E87 |
| | E87S, E87T, E87H | |
| H108 | H93G, H93A | H93 |
| N110 | N95V, N95A, N95S, N95T | N95 |
| S114 | S99A | S99 |
| K116 | K101E | K101 |
| H117 | H102Y, H102A | H102 |
| W122 | W107A | W107 |
| F123 | F108Y | F108 |
| S131 | S116R | S116 |
| C132 | C117V, C117P, C117T, | C117 |
| | C117S, C117A | |
| F147 | F132W | F132 |

In some examples, the mutant FGF1 protein includes mutations at one or more of the following positions: K12, H21, Q40, L44, S47, H93, N95, H102, and F108 such as 1 to 5, 2 to 5, 3 to 6, 3 to 5, 3 to 8, 1, 2, 3, 4, 5, 6, 7, 8, 9, 10, 11, 12, 13, 14, 15, 16, 17, 18, or 19, of these positions.

In some examples, the mutant FGF1 protein includes mutations at 1, 2, 3, or 4, of the following positions: K12, A66, N95, and C117 (wherein the numbering refers to SEQ ID NO: 5), such as one or more of K12V, A66C, N95V, and C117V, (such as 1, 2, 3, or 4 of these mutations).

In some examples, the mutant FGF1 protein includes mutations at 1, 2, 3, or 4, of the following positions: S99, K101, H102, and W107 (wherein the numbering refers to SEQ ID NO: 5), such as one or more of S99A, K101E, H102A, and W107A, (such as 1, 2, 3, or 4 of these mutations).

In one example, the mutant FGF1 protein includes a mutation at E87 or N95, such as replacement with a non-charged amino acid.

In some examples, the mutant FGF1 protein includes a mutation at K12 of FGF1, which is predicted to be at the receptor interface. Thus, K12 of SEQ ID NO: 5 can be mutated, for example to a V or C.

In some examples, the mutant FGF1 protein includes at least 90 consecutive amino acids from amino acids 5-141 of FGF1 (e.g., of SEQ ID NOS: 2 or 4), (which in some examples can include further deletion of N-terminal amino acids 1-20 and/or point mutations, such as substitutions, deletions, and/or additions). In some examples, the mutant FGF1 protein includes at least 100 or at least 110 consecutive amino acids from amino acids 5-141 of FGF1, such as at least 100 consecutive amino acids from amino acids 5-141 of SEQ ID NO: 2 or 4 or at least 100 consecutive amino acids from SEQ ID NO: 5.

In some examples, the mutant FGF1 protein includes both an N-terminal truncation and additional point mutations. Specific exemplary FGF1 mutant proteins are shown in SEQ ID NOS: 10-25. In some examples, the FGF1 mutant includes an N-terminal deletion, but retains a methionine at the N-terminal position. In some examples, the FGF1 mutant is 120-140 or 125-140 amino acids in length.

In some examples, the FGF1 mutant protein includes at least 80% sequence identity to any of one SEQ ID NOs: 10-25. Thus, the FGF1 mutant protein can have at least 85%, at least 90%, at least 95%, at least 96%, at least 97%, at least 98% or at least 99% sequence identity to any of one SEQ ID NOS: 10-25 (but is not a native FGF1 sequence, such as SEQ ID NO: 5). In some examples, the FGF1 mutant protein includes or consists of any of one SEQ ID NOS: 10-25. The disclosure encompasses variants of the disclosed FGF1 mutant proteins, such as any of one SEQ ID NOS: 10-25 having 1 to 8, 2 to 10, 1 to 5, 1 to 6, or 5 to 10 additional mutations, such as conservative amino acid substitutions.

Also provided are isolated nucleic acid molecules encoding the disclosed mutated FGF1 proteins, such as a nucleic acid molecule encoding a protein having at least 80%, at least 85%, at least 90%, at least 95%, at least 96%, at least 97%, at least 98%, or at least 99% sequence identity to any of one SEQ ID NOS: 10-25 (but is not a native FGF1 sequence). Vectors and cells that include such nucleic acid molecules are also provided. For example, such nucleic acid molecules can be expressed in a host cell, such as a bacterium or yeast cell (e.g., E. coli), thereby permitting expression of the mutated FGF1 protein. The resulting mutated FGF1 protein can be purified from the cell.

Methods of using the disclosed mutated FGF1 proteins are provided. As discussed herein, the mutated mature FGF1 protein can include a deletion of at least six contiguous N-terminal amino acids, at least one additional point mutation, or combinations thereof. For example, such methods include administering a therapeutically effective amount of a disclosed mutated FGF1 protein (such as at least 0.01 mg/kg, at least 0.05 mg/kg, at least 0.1 mg/kg, or at least 0.5 mg/kg) (or nucleic acid molecules encoding such) to reduce blood glucose in a mammal, such as a decrease of at least 5%, at least 10%, at least 25% or at least 50%, for example as compared to administration of no mutant FGF1 mutant protein (e.g., administration of PBS).

In one example, the method is a method of reducing fed and fasting blood glucose, improving insulin sensitivity and glucose tolerance, reducing systemic chronic inflammation, ameliorating hepatic steatosis in a mammal, reducing triglycerides, decreasing insulin resistance, reducing hyperinsulinemia, increasing glucose tolerance, reducing hyperglycemia, reducing food intake, or combinations thereof. Such a method can include administering a therapeutically effective amount of one or more disclosed mutated FGF1 proteins (such as at least 0.01 mg/kg, at least 0.05 mg/kg, at least 0.1 mg/kg, or at least 0.5 mg/kg) (or nucleic acid molecules encoding such) to reduce fed and fasting blood glucose, improve insulin sensitivity and glucose tolerance, reduce systemic chronic inflammation, ameliorate hepatic steatosis in a mammal, reduce food intake, or combinations thereof.

In one example, the method is a method of treating a metabolic disease (such as metabolic syndrome, diabetes, or obesity) in a mammal Such a method can include administering a therapeutically effective amount of one or more disclosed mutated FGF1 proteins (such as at least 0.01 mg/kg, at least 0.05 mg/kg, at least 0.1 mg/kg, or at least 0.5 mg/kg) (or nucleic acid molecules encoding such) to treat the metabolic disease.

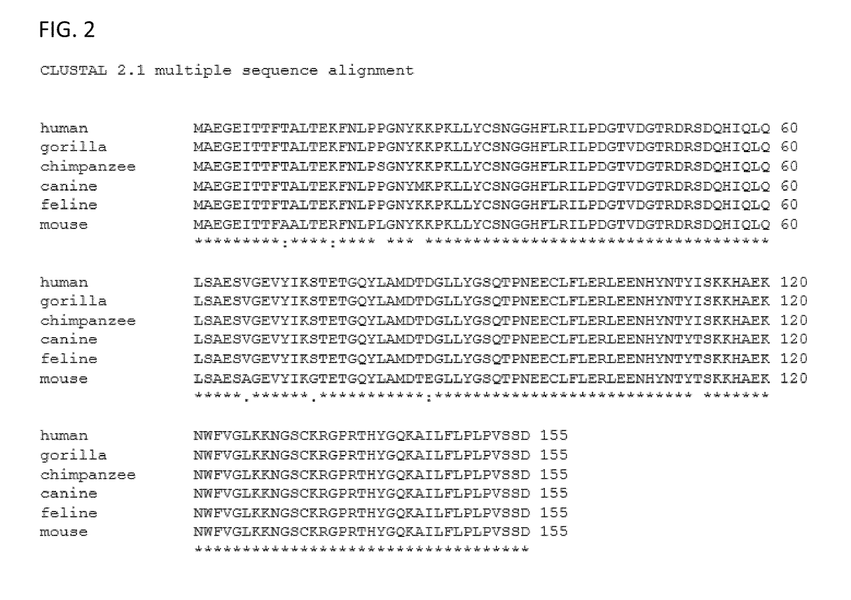

In some examples, the mammal, such as a human, cat, or dog, has diabetes. Methods of administration are routine, and can include subcutaneous, intraperitoneal, intramuscular, or intravenous injection or infusion. In some examples, the mutated FGF1 protein is a mutated canine FGF1 protein, and is used to treat a dog. For example, a canine FGF1 (such as XP_849274.1) can be mutated to include an S131 mutation (referring to amino acid 131 in XP_849274.1), such as S131R, which is analogous to the human S116R mutation. This mutation can also be used in combination with, for example, an N-terminal deletion, and/or one or more additional point mutations. Similarly, in some embodiments, the mutated FGF1 protein containing an S116 mutation (such as S116R) is a mutated cat FGF1 protein, and is used to treat a cat. Thus, for example, a feline FGF1 (such as XP_011281008.1) can be mutated to include an S131 mutation (which is amino acid 131 in XP_011281008.1), such as S131R, and can also be used in combination with an N-terminal deletion and/or one or more additional point mutations. Based on routine methods of sequence alignment (e.g., see FIG. 2), one skilled in the art can mutate any known FGF1 sequence to generate mutations that correspond to those provided herein (for example, the FGF1 sequence can be selected based on the subject to be treated, e.g., a dog can be treated with a mutated canine FGF1 protein or corresponding nucleic acid molecule).

In some examples, use of the FGF1 mutants disclosed herein does not lead to (or significantly reduces, such as a reduction of at least 20%, at least 50%, at least 75%, or at least 90%) the adverse side effects observed with thiazolidinediones (TZDs) therapeutic insulin sensitizers, including weight gain, increased liver steatosis and bone fractures (e.g., reduced effects on bone mineral density, trabecular bone architecture and cortical bone thickness).

Provided are methods of reducing fed and fasting blood glucose, improving insulin sensitivity and glucose tolerance, reducing systemic chronic inflammation, ameliorating hepatic steatosis, reducing food intake, or combinations thereof, in a mammal, such as within 12 hours, within 24 hours, or within 48 hours of the treatment, such as within 12 to 24 hours, within 12 to 36 hours, or within 24 to 48 hours. Such methods can include administering a therapeutically effective amount of a FGF1 mutant disclosed herein, to the mammal, or a nucleic acid molecule encoding the FGF1 mutant or a vector comprising the nucleic acid molecule, thereby reducing fed and fasting blood glucose, improving insulin sensitivity and glucose tolerance, reducing systemic chronic inflammation, ameliorating hepatic steatosis, reduce one or more non-HDL lipid levels, reduce food intake, or combinations thereof, in a mammal. In some examples, the fed and fasting blood glucose is reduced in the treated subject by at least 10%, at least 20%, at least 30%, at least 50%, at least 75%, or at least 90% as compared to an absence of administration of the FGF1 mutant. In some examples, insulin sensitivity and glucose tolerance is increased in the treated subject by at least 10%, at least 20%, at least 30%, at least 50%, at least 75%, or at least 90% as compared to an absence of administration of the FGF1 mutant. In some examples, systemic chronic inflammation is reduced in the treated subject by at least 10%, at least 20%, at least 30%, at least 50%, at least 75%, or at least 90% as compared to an absence of administration of the FGF1 mutant. In some examples, hepatic steatosis is reduced in the treated subject by at least 10%, at least 20%, at least 30%, at least 50%, at least 75%, or at least 90% as compared to an absence of administration of the FGF1 mutant. In some examples, one or more lipids (such as a non-HDL, for example IDL, LDL and/or VLDL) are reduced in the treated subject by at least 10%, at least 20%, at least 30%, at least 50%, at least 75%, or at least 90% as compared to an absence of administration of the FGF1 mutant. In some examples, triglyceride and or cholesterol levels are reduced with the FGF1 mutant by at least 10%, at least 20%, at least 30%, at least 50%, at least 75%, or at least 90% as compared to an absence of administration of the FGF1 mutant. In some examples, the amount of food intake is reduced in the treated subject by at least 10%, at least 20%, at least 30%, at least 50%, at least 75%, or at least 90% as compared to an absence of administration of the FGF1 mutant (such as within 12 hours, within 24 hours, or within 48 hours of the treatment, such as within 12 to 24 hours, within 12 to 36 hours, or within 24 to 48 hours). In some examples, combinations of these reductions are achieved.

### Mutated FGF1 Proteins

The present disclosure provides mutated FGF1 proteins. Such mutants include an N-terminal deletion, one or more point mutations (such as amino acid substitutions, deletions, additions, or combinations thereof), or combinations of N-terminal deletions and one or more additional point mutations. Such proteins and corresponding coding sequences can be used in the methods provided herein.

In some examples, the disclosed FGF1 mutant proteins have reduced mitogenicity compared to mature native FGF1 (e.g., SEQ ID NO: 5), such as a reduction of at least 20%, at least 50%, at least 75% or at least 90%.

In one example, the disclosed FGF1 mutant proteins have improved thermostability compared to mature native FGF1 (e.g., SEQ ID NO: 5), such as an increase of at least 10%, at least 20%, at least 50%, or at least 75% (e.g., see Xia et al., PLoS One. 2012; 7(11):e48210 and Zakrzewska, J Biol Chem. 284:25388-25403, 2009). Methods of measuring FGF1 stability are known in the art, such as measuring denaturation of FGF1 or mutants by fluorescence and circular dichroism in the absence and presence of a 5-fold molar excess of heparin in the presence of 1.5 M urea or isothermal equilibrium denaturation by guanidine hydrochloride. In one example, the assay provided by Dubey et al., J. Mol. Biol. 371:256-268, 2007 is used to measure FGF1 stability.

In one example, the disclosed FGF1 mutant proteins have improved protease resistance compared to mature native FGF1 (e.g., SEQ ID NO: 5), such as an increase of at least 10%, at least 20%, at least 50%, or at least 75% (e.g., see Kobielak et al., Protein Pept Lett. 21(5):434-43, 2014).

In some examples, the mutant FGF1 is a truncated version of the mature protein (e.g., SEQ ID NO: 5), which can include for example deletion of at least 5, at least 6, at least 9, at least 10, at least 11, at least 12, at least 13, at least 14, at least 15, or at least 20 consecutive N-terminal amino acids. Thus, in some examples, the mutant FGF1 protein is a truncated version of the mature protein (e.g., SEQ ID NO: 5), such a deletion of the N-terminal 5, 6, 7, 8, 9, 10, 11, 12, 13, 14, 15, 16, 17, 18, 19 or 20 amino acids shown in SEQ ID NO: 5. Examples of N-terminally truncated FGF1 proteins are shown in SEQ ID NOS: 13, 14, 15, 16, 17, 18, 19, 20, 21, 22, 23, and 24. In some examples, the FGF1 mutant includes an N-terminal deletion, but retains a methionine at the N-terminal position. In some examples, such an N-terminally deleted FGF1 protein has reduced mitogenic activity as compared to wild-type mature FGF1 protein. In some examples, such an N-terminally deleted FGF1 protein has amino acids added to the N-terminus, such as adding the sequence MRDSSPL (e.g., see SEQ ID NOS: 13, 14, 15, 20 and 21).

Thus, in some examples, the mutant FGF1 protein includes at least 90 consecutive amino acids from amino acids 5-141 or 5-155 of FGF1 (e.g., of SEQ ID NOS: 2 or 4), (which in some examples can include further deletion of N-terminal amino acids 1-20 and/or point mutations, such as substitutions, deletions, and/or additions). In some examples, the mutant FGF1 protein includes at least 90 consecutive amino acids from amino acids 1-140 of FGF1 (e.g., of SEQ ID NO: 5), (which in some examples can include further deletion of N-terminal amino acids 1-20 and/or point mutations, such as substitutions, deletions, and/or additions). Thus, in some examples, the mutant FGF1 protein includes at least 90 consecutive amino acids from amino acids 5-141 of FGF1, such as at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 115, at least 120, at least 125, or at least 130 consecutive amino acids from amino acids 5-141 of SEQ ID NOS: 2 or 4 (such as 90-115, 90-125, 90-100, or 90-95 consecutive amino acids from amino acids 5-141 of SEQ ID NOS: 2 or 4). In some examples, the mutant FGF1 protein includes least 90 consecutive amino acids from SEQ ID NO: 5. Thus, in some examples, the mutant FGF1 protein includes at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, or at least 110 consecutive amino acids from SEQ ID NO: 5 (such as 90-115, 90-100, or 90-95 consecutive amino acids from SEQ ID NO: 5).

In some examples, the mutant FGF1 protein includes at least 1, at least 4, at least 5, at least 6, at least 7, at least 8, at least 9, at least 10, at least 11, at least 12, at least 13, at least 14, at least 15, at least 16, at least 17, at least 18, at least 19, or at least 20 additional amino acid substitutions, such as 1-20, 1-10, 4-8, 5-12, 5-10, 5-25, 1, 2, 3, 4, 5, 6, 7, 8, 9, 10, 11, 12, 13, 14, or 15 additional amino acid substitutions. For example, point mutations can be introduced into an FGF1 sequence to decrease mitogenicity, increase stability, alter binding affinity for heparin and/or heparan sulfate (compared to the portion of a native FGF1 protein without the modification), or combinations thereof. Specific exemplary point mutations that can be used are shown above in Table 1.

In some examples, the mutant FGF1 protein includes one or more mutations (such as a substitution or deletion) at one or more of the following positions: K12, H21, Q40, L44, S47, Y55, A66, C83, E87, H93, N95, S99, K101, H102, W107, F108, S116, C117, and F132, such as 1, 2, 3, 4, 5, 6, 7, 8, 9, 10, 11, 12, 13, 14, 15, 16, 17, 18, or 19 of these positions. In some examples the mutant FGF1 protein has as one or more of K12V, H21Y, Q40K, L44F, S47A, S47V, S47I, Y55F, Y55V, Y55S, Y55A, Y55W, A66C, C83T, C83S, C83A, C83V, E87Q, E87D, E87V, E87A, E87S, E87T, E87H, H93G, H93A, N95V, N95A, N95S, N95T, S99A, K101E, H102Y, H102A, W107A, F108Y, S116R, C117V, C117P, C117T, C117S, C117A, and F132W (wherein the numbering refers to SEQ ID NO: 5), such as 1 to 5, 1 to 10, 2 to 5, 2 to 10, 3 to 6, or 2 to 8 of these mutations, such as 1, 2, 3, 4, 5, 6, 7, 8, 9, 10, 11, 12, 13, 14, 15, 16, 17, 18, or 19 of these mutations.

In some examples, the mutant FGF1 protein includes both an N-terminal truncation and one or more additional point mutations. Specific exemplary FGF1 mutant proteins are shown in SEQ ID NOS: 10-25. In some examples, the FGF1 mutant protein includes at least 80% sequence identity to any of SEQ ID NOS: 10-25. Thus, the FGF1 mutant protein can have at least 85%, at least 90%, at least 95%, at least 96%, at least 97%, at least 98% or at least 99% sequence identity to any of SEQ ID NOS: 10-25. In some examples, the FGF1 mutant protein includes or consists of any of SEQ ID NOS: 10-25. The disclosure encompasses variants of the disclosed FGF1 mutant proteins, such as variants of any of SEQ ID NOS: 10-25 having 1 to 20, 1 to 15, 1 to 10, 1 to 8, 2 to 10, 1 to 5, 1 to 6, 2 to 12, 3 to 12, 5 to 12, or 5 to 10 additional mutations, such as conservative amino acid substitutions.

In some examples, the mutant FGF1 protein has at its N-terminus a methionine. In some examples, the mutant FGF1 protein is at least 120 amino acids in length, such as at least 125, at least 130, at least 135, at least 140, at least 145, at least 150, at least 155, at least 160, or at least 175 amino acids in length, such as 120-160, 125-160, 130-160, 150-160, 130-200, 130-180, 130-170, or 120-160 amino acids in length.

Exemplary mutant FGF1 proteins are provided in SEQ ID NOS: 10-25. One skilled in the art will recognize that minor variations can be made to these sequences, without adversely affecting the function of the protein (such as its ability to reduce blood glucose). For example, variants of the mutant FGF1 proteins include those having at least 80%, at least 85%, at least 90%, at least 95%, at least 96%, at least 97%, at least 98%, or at least 99% sequence identity to any one of SEQ ID NOS: 10-25 (but are not a native FGF1 sequence, e.g., SEQ ID NO: 5), but retain the ability to treat a metabolic disease, or decrease blood glucose in a mammal (such as a mammal with type II diabetes). Thus, variants of any one of SEQ ID NOS: 10-25 retaining at least 80%, at least 85%, at least 90%, at least 95%, at least 96%, at least 97%, at least 98%, or at least 99% sequence identity are of use in the disclosed methods.

### FGF1

FGF1 (such as SEQ ID NOS: 2, 4 or 5) can be mutated to include mutations to control (e.g., reduce) the mitogenicity of the protein and to provide glucose-lowering ability to the protein. Mutations can also be introduced to affect the stability and receptor binding selectivity of the protein.

Exemplary full-length FGF1 proteins are shown in SEQ ID NOS: 2 (human) and 4 (mouse). In some examples, FGF1 includes SEQ ID NO: 2 or 4, but without the N-terminal methionine (resulting in a 154 aa FGF1 protein). In addition, the mature/active form of FGF1 is one where a portion of the N-terminus is removed, such as the N-terminal 15, 16, 20, or 21 amino acids from SEQ ID NO: 2 or 4. Thus, in some examples the active form of FGF1 comprises or consists of amino acids 16-155 or 22-155 of SEQ ID NOS: 2 or 4 (e.g., see SEQ ID NO: 5). In some examples, the mature form of FGF1 that can be mutated includes SEQ ID NO: 5 with a methionine added to the N-terminus (wherein such a sequence can be mutated as discussed herein). Thus, a mutated mature FGF1 protein can include an N-terminal truncation.

In some examples, multiple types of mutations disclosed herein are made to an FGF1 protein. Although mutations below are noted by a particular amino acid for example in SEQ ID NOS: 2, 4, or 5, one skilled in the art will appreciate that the corresponding amino acid can be mutated in any FGF1 sequence. For example, Q40 of SEQ ID NO: 5 corresponds to Q55 of SEQ ID NOS: 2 and 4.

In one example, mutations are made to the N-terminal region of FGF1 (such as SEQ ID NOS: 2, 4 or 5), such as deletion of the first 14, 15, 16, 17, 18, 19, 20, 21, 22, 23, 24, 25, 26, 27, 28, 29, or 30 amino acids of SEQ ID NOS: 2 or 4 (such as deletion of at least the first 14 amino acids of SEQ ID NO: 2 or 4, such as deletion of at least the first 15, at least 16, at least 20, at least 25, or at least 29 amino acids of SEQ ID NOS: 2 or 4), deletion of the first 8, 9, 10, 11, 12, 13, 14, 15, 16, 17, 18, 19, or 20 amino acids of SEQ ID NO: 5 (e.g., see SEQ ID NOS: 13-24).

Mutations can be made to a mutant FGF1 (such as to any of SEQ ID NOS: 10-25) to reduce its mitogenic activity. In some examples, such mutations reduce mitogenic activity by at least 50%, at least 60%, at least 70%, at least 75%, at least 80%, at least 90%, at least 92%, at least 95%, at least 98%, at least 99%, or even complete elimination of detectable mitogenic activity, as compared to a native FGF1 protein without the mutation. Methods of measuring mitogenic activity are known in the art, such as thymidine incorporation into DNA in serum-starved cells (e.g., NIH 3T3 cells) stimulated with the mutated FGF1, methylthiazoletetrazolium (MTT) assay (for example by stimulating serum-starved cells with mutated FGF1 for 24 hr then measuring viable cells), cell number quantification or BrdU incorporation. In some examples, the assay provided by Fu et al., World J. Gastroenterol. 10:3590-6, 2004; Klingenberg et al., J. Biol. Chem. 274:18081-6, 1999; Shen et al., Protein Expr Purif. 81:119-25, 2011, or Zou et al., Chin. Med. J. 121:424-429, 2008 is used to measure mitogenic activity.

Mutations that reduce the heparan binding affinity (such as a reduction of at least 10%, at least 20%, at least 50%, or at least 75%, e.g., as compared to a native FGF1 protein without the mutation), can also be used to reduce mitogenic activity, for example by substituting heparan binding residues from a paracrine FGFs into a mutant FGF1.

In some examples, an FGF1 mutant includes mutations to the FGF1 nuclear export sequence, for example to decrease the amount of FGF1 in the nucleus and reduce its mitogenicity as measured by thymidine incorporation assays in cultured cells (e.g., see Nilsen et al., J. Biol. Chem. 282(36):26245-56, 2007). Mutations to the nuclear export sequence decrease FGF1-induced proliferation (e.g., see Nilsen et al., J. Biol. Chem. 282(36):26245-56, 2007). Methods of measuring FGF1 degradation are known in the art, such as measuring [³⁵S]methionine-labeled FGF1 or immunoblotting for steady-state levels of FGF1 in the presence or absence of proteasome inhibitors. In one example, the assay provided by Nilsen et al., J. Biol. Chem. 282(36):26245-56, 2007 or Zakrzewska et al., J. Biol. Chem. 284:25388-403, 2009 is used to measure FGF1 degradation.

In some examples, the mutant FGF1 protein is PEGylated at one or more positions, such as at N95 (for example see methods of Niu et al., J. Chromatog. 1327:66-72, 2014, herein incorporated by reference). Pegylation consists of covalently linking a polyethylene glycol group to surface residues and/or the N-terminal amino group. N95 is known to be involved in receptor binding, and thus, is on the surface of the folded protein. As mutations to surface exposed residues could potentially generate immunogenic sequences, pegylation is an alternative method to abrogate a specific interaction. Pegylation is an option for any surface exposed site implicated in the receptor binding and/or proteolytic degradation. Pegylation can “cover” functional amino acids, e.g. N95, as well as increase serum stability.

In some examples, the mutant FGF1 protein includes an immunoglobin FC domain (for example see Czajkowsky et al., EMBO Mol. Med. 4:1015-28, 2012, herein incorporated by reference). The conserved FC fragment of an antibody can be incorporated either N-terminal or C-terminal of the mutant FGF1 protein, and can enhance stability of the protein and therefore serum half-life. The FC domain can also be used as a means to purify the proteins on Protein A or Protein G sepharose beads. This makes the FGF1 mutants having heparin binding mutations easier to purify.

### Variant Sequences

Variant mutant FGF1 proteins, including variants of the sequences shown in Table 1, and variants of any one of SEQ ID NOS: 10-25, can contain one or more mutations, such as a single insertion, a single deletion, a single substitution. In some examples, the mutant FGF1 protein includes 1-20 insertions, 1-20 deletions, 1-20 substitutions, and/or any combination thereof (e.g., single insertion together with 1-19 substitutions). In some examples, the disclosure provides a variant of any disclosed mutant FGF1 protein having 1, 2, 3, 4, 5, 6, 7, 8, 9, 10, 11, 12, 13, 14, 15, 16, 17, 18, 19 or 20 additional amino acid changes. In some examples, any one of SEQ ID NOS: 10-25, includes 1-8 insertions, 1-15 deletions, 1-10 substitutions, and/or any combination thereof (e.g., 1-15, 1-4, or 1-5 amino acid deletions together with 1-10, 1-5 or 1-7 amino acid substitutions). In some examples, the disclosure provides a variant of any one of SEQ ID NOS: 10-25, having 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes. In one example, such variant peptides are produced by manipulating the nucleotide sequence encoding a peptide using standard procedures such as site-directed mutagenesis or PCR. Such variants can also be chemically synthesized.

One type of modification or mutation includes the substitution of amino acids for amino acid residues having a similar biochemical property, that is, a conservative substitution (such as 1-4, 1-8, 1-10, or 1-20 conservative substitutions). Typically, conservative substitutions have little to no impact on the activity of a resulting peptide. For example, a conservative substitution is an amino acid substitution in any one of SEQ ID NOS: 10-25, that does not substantially affect the ability of the peptide to decrease blood glucose in a mammal. An alanine scan can be used to identify which amino acid residues in a mutant FGF1 protein, such as any one of SEQ ID NOS: 10-25, can tolerate an amino acid substitution. In one example, the blood glucose lowering activity of FGF1, or any one of SEQ ID NOS: 10-25 is not altered by more than 25%, for example not more than 20%, for example not more than 10%, when an alanine, or other conservative amino acid, is substituted for 1-4, 1-8, 1-10, or 1-20 native amino acids. Examples of amino acids which may be substituted for an original amino acid in a protein and which are regarded as conservative substitutions include: Ser for Ala; Lys, Gln, or Asn for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions and/or additions) can be assessed by analyzing the function of the mutant FGF1 protein, such as any one of SEQ ID NOS: 10-25, by analyzing the ability of the variant protein to decrease blood glucose in a mammal.

### Generation of Proteins

Isolation and purification of recombinantly expressed mutated FGF1 proteins can be carried out by conventional means, such as preparative chromatography and immunological separations. Once expressed, mutated FGF1 proteins can be purified according to standard procedures of the art, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, mutated FGF1 proteins disclosed herein can also be constructed in whole or in part using standard peptide synthesis. In one example, mutated FGF1 proteins are synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### Mutated FGF1 Nucleic Acid Molecules and Vectors

Nucleic acid molecules encoding a mutated FGF1 protein are encompassed by this disclosure. Based on the genetic code, nucleic acid sequences coding for any mutated FGF1 sequence, such as those generated using the mutations shown in Table 1, can be routinely generated. In some examples, such a sequence is optimized for expression in a host cell, such as a host cell used to express the mutant FGF1 protein.

In one example, a nucleic acid sequence codes for a mutant FGF1 protein having at least 60%, at least 70%, at least 75%, at least 80%, at least 90%, at least 92%, at least 95%, at least 96%, at least 97%, at least 98%, or at least 99% sequence identity to any one of SEQ ID NOS: 10-25, can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same mutant FGF1 protein sequence.

Nucleic acid molecules include DNA, cDNA, and RNA sequences which encode a mutated FGF1 peptide. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) that take advantage of the codon usage preferences of that particular species. For example, the mutated FGF1 proteins disclosed herein can be designed to have codons that are preferentially used by a particular organism of interest.

A nucleic acid encoding a mutant FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). A wide variety of cloning and in vitro amplification methodologies are well known to persons skilled in the art. In addition, nucleic acids encoding sequences encoding a mutant FGF1 (such as one encoding a protein generated using the mutations shown in Table 1, the sequences in any one of SEQ ID NOs: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be prepared by cloning techniques. Examples of appropriate cloning and sequencing techniques, and instructions sufficient to direct persons of skill through cloning are found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

In one example, a mutant FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOs: 10-25) is prepared by inserting the cDNA which encodes the mutant FGF1 protein into a vector. The insertion can be made so that the mutant FGF1 protein is read in frame so that the mutant FGF1 protein is produced.

The mutated FGF1 nucleic acid coding sequence (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect, plant, and mammalian organisms. Methods of expressing DNA sequences having eukaryotic or viral sequences in prokaryotes are well known in the art. Biologically functional viral and plasmid DNA vectors capable of expression and replication in a host are known in the art. The vector can encode a selectable marker, such as a thymidine kinase gene.

Nucleic acid sequences encoding a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOs: 10-25) can be operatively linked to expression control sequences. An expression control sequence operatively linked to a mutated FGF1 protein coding sequence is ligated such that expression of the mutant FGF1 protein coding sequence is achieved under conditions compatible with the expression control sequences. The expression control sequences include, but are not limited to appropriate promoters, enhancers, transcription terminators, a start codon (i.e., ATG) in front of a mutated FGF1 protein-encoding gene, splicing signal for introns, maintenance of the correct reading frame of that gene to permit proper translation of mRNA, and stop codons.

In one embodiment, vectors are used for expression in yeast such as S. cerevisiae, P. pastoris, or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding a mutated FGF1 protein (such as one encoding a protein generated using the mutations shown in Table 1, the sequences in any one of SEQ ID NOs: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can also be designed to express in insect cells.

A mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared that encode a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25). Exemplary viral vectors include polyoma, SV40, adenovirus, vaccinia virus, adeno-associated virus, herpes viruses including HSV and EBV, Sindbis viruses, alphaviruses and retroviruses of avian, murine, and human origin. Baculovirus (Autographa californica multinuclear polyhedrosis virus; AcMNPV) vectors are also known in the art, and may be obtained from commercial sources. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, and poliovirus vectors. Specific exemplary vectors are poxvirus vectors such as vaccinia virus, fowlpox virus and a highly attenuated vaccinia virus (MVA), adenovirus, baculovirus, and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox, and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that encode a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can include at least one expression control element operationally linked to the nucleic acid sequence encoding the mutated FGF1 protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the mutated FGF1 protein in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding the mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) are known. Such techniques involve, for example, homologous recombination between the viral DNA sequences flanking the DNA sequence in a donor plasmid and homologous sequences present in the parental virus. The vector can be constructed for example by steps known in the art, such as by using a unique restriction endonuclease site that is naturally present or artificially inserted in the parental viral vector to insert the heterologous DNA.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression system such as plasmids and vectors of use in producing mutated FGF1 proteins in cells including higher eukaryotic cells such as the COS, CHO, HeLa and myeloma cell lines.

### Cells Expressing Mutated FGF1 Proteins

A nucleic acid molecule encoding a mutated FGF1 protein disclosed herein can be used to transform cells and make transformed cells. Thus, cells expressing a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), are disclosed. Cells expressing a mutated FGF1 protein disclosed herein can be eukaryotic or prokaryotic. Examples of such cells include, but are not limited to bacteria, archea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, SF9 cells, C129 cells, 293 cells, Neurospora, and immortalized mammalian myeloid and lymphoid cell lines.

Cells expressing a mutated FGF1 protein are transformed or recombinant cells. Such cells can include at least one exogenous nucleic acid molecule that encodes a mutated FGF1 protein, for example one encoding a protein generated using the mutations shown in Table 1, the sequences in any of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25. It is understood that all progeny may not be identical to the parental cell since there may be mutations that occur during replication. Methods of stable transfer, meaning that the foreign DNA is continuously maintained in the host cell, are known in the art.

Transformation of a host cell with recombinant DNA may be carried out by conventional techniques as are well known. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation. Techniques for the propagation of mammalian cells in culture are well-known (see, Jakoby and Pastan (eds.), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. Techniques for the transformation of yeast cells, such as polyethylene glycol transformation, protoplast transformation, and gene guns are also known in the art.

### Pharmaceutical Compositions that Include Mutated FGF1 Molecules

Pharmaceutical compositions that include a mutated FGF1 protein (such as one encoding a protein generated using the mutations shown in Table 1, the sequences in any one of SEQ ID NOS: 10-25, or those encoding a protein having at least at least 80%, at least 85%, 90%, at least 92%, at least 95%, at least 96%, at least 97%, at least 98%, at least 99%, or 100% sequence identity to any one of SEQ ID NOS: 10-25) or a nucleic acid encoding these proteins, can be formulated with an appropriate pharmaceutically acceptable carrier, depending upon the particular mode of administration chosen.

In some embodiments, the pharmaceutical composition consists essentially of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) (or a nucleic acid encoding such a protein) and a pharmaceutically acceptable carrier. In these embodiments, additional therapeutically effective agents are not included in the compositions.

In other embodiments, the pharmaceutical composition includes a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) (or a nucleic acid encoding such a protein) and a pharmaceutically acceptable carrier. Additional therapeutic agents, such as agents for the treatment of diabetes, can be included. Thus, the pharmaceutical compositions can include a therapeutically effective amount of another agent. Examples of such agents include, without limitation, anti-apoptotic substances such as the Nemo-Binding Domain and compounds that induce proliferation such as cyclin dependent kinase (CDK)-6, CDK-4 and cyclin D1. Other active agents can be utilized, such as antidiabetic agents for example, insulin, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570, aleglitazar, farglitazar, muraglitazar, tesaglitazar, and TZD) and PPAR-γ antagonists, PPAR-gamma/alpha modulators (such as KRP 297), alpha-glucosidase inhibitors (e.g., acarbose, voglibose), dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g. exendin-4) or amylin. Additional examples include immunomodulatory factors such as anti-CD3 mAb, growth factors such as HGF, VEGF, PDGF, lactogens, and PTHrP. In some examples, the pharmaceutical compositions containing a mutated FGF1 protein can further include a therapeutically effective amount of other FGFs, such as FGF21, FGF19, or both, heparin, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other embodiments, a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) is included in a nanodispersion system. Nanodispersion systems and methods for producing such nanodispersions are well known to one of skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

With regard to the administration of nucleic acids, one approach to administration of nucleic acids is direct treatment with plasmid DNA, such as with a mammalian expression plasmid. As described above, the nucleotide sequence encoding a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be placed under the control of a promoter to increase expression of the protein.

Many types of release delivery systems are available and known. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or polynucleotide encoding this protein, is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant may be particularly suitable for treatment of chronic conditions, such as diabetes. Long-term release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. These systems have been described for use with nucleic acids (see U.S. Pat. No. 6,218,371). For use in vivo, nucleic acids and peptides are preferably relatively resistant to degradation (such as via endo- and exo-nucleases). Thus, modifications of the disclosed mutated FGF1 proteins, such as the inclusion of a C-terminal amide, can be used.

The dosage form of the pharmaceutical composition can be determined by the mode of administration chosen. For instance, in addition to injectable fluids, topical, inhalation, oral, and suppository formulations can be employed. Topical preparations can include eye drops, ointments, sprays, patches, and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutical compositions that include a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be formulated in unit dosage form, suitable for individual administration of precise dosages. In one non-limiting example, a unit dosage contains from about 1 mg to about 1 g of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), such as about 10 mg to about 100 mg, about 50 mg to about 500 mg, about 100 mg to about 900 mg, about 250 mg to about 750 mg, or about 400 mg to about 600 mg. In other examples, a therapeutically effective amount of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) is about 0.01 mg/kg to about 50 mg/kg, for example, about 0.5 mg/kg to about 25 mg/kg, about 0.05 mg/kg to about 0.1 mg/kg, about 0.01 mg/kg to about 0.1 mg/kg, or about 1 mg/kg to about 10 mg/kg. In other examples, a therapeutically effective amount of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) is about 1 mg/kg to about 5 mg/kg, for example about 2 mg/kg. In a particular example, a therapeutically effective amount of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) includes about 1 mg/kg to about 10 mg/kg, such as about 2 mg/kg. In a particular example, a therapeutically effective amount of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) includes about 0.01 mg/kg to about 0.5 mg/kg, such as about 0.1 mg/kg.

### Treatment Using a Mutated FGF1 Protein

The disclosed mutated FGF1 proteins (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or nucleic acids encoding such proteins, can be administered to a subject, for example to treat a metabolic disease, for example by reducing fed and fasting blood glucose, improving insulin sensitivity and glucose tolerance, reducing systemic chronic inflammation, ameliorating hepatic steatosis in a mammal, reducing food intake, or combinations thereof.

The compositions of this disclosure that include a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) (or nucleic acids encoding these molecules) can be administered to humans or other animals by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering a mutated FGF1 protein such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) (or a nucleic acid encoding these molecules) to pancreas tissue (for example by using a pump, or by implantation of a slow release form at the site of the pancreas). The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly, every other week, monthly, every 7 days, every 10 days, every 14 days, every 30 days, etc.) doses over a period of a few days, few weeks, to months, or even years. For example, a therapeutically effective amount of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be administered in a single dose, twice daily, weekly, every other week, or in several doses, for example daily, or during a course of treatment. In a particular non-limiting example, treatment involves once daily dose, twice daily dose, once weekly dose, every other week dose, or monthly dose.

The amount of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOs: 10-25) administered can be dependent on the subject being treated, the severity of the affliction, and the manner of administration, and is best left to the judgment of the prescribing clinician. Determination of the appropriate amount to be administered is within the routine level of ordinary skill in the art. Within these bounds, the formulation to be administered will contain a quantity of the mutated FGF1 protein in amounts effective to achieve the desired effect in the subject being treated. A therapeutically effective amount of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be the amount of the mutant FGF1 protein or a nucleic acid encoding these molecules that is necessary to treat diabetes or reduce blood glucose levels (for example a reduction of at least 5%, at least 10% or at least 20%, for example relative to no administration of the mutant FGF1).

When a viral vector is utilized for administration of an nucleic acid encoding a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), the recipient can receive a dosage of each recombinant virus in the composition in the range of from about 10⁵ to about 10¹⁰ plaque forming units/mg mammal, although a lower or higher dose can be administered. Examples of methods for administering the composition into mammals include, but are not limited to, exposure of cells to the recombinant virus ex vivo, or injection of the composition into the affected tissue or intravenous, subcutaneous, intradermal, or intramuscular administration of the virus. Alternatively the recombinant viral vector or combination of recombinant viral vectors may be administered locally by direct injection into the pancreas in a pharmaceutically acceptable carrier.

Generally, the quantity of recombinant viral vector, carrying the nucleic acid sequence of the mutated FGF1 protein to be administered (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) is based on the titer of virus particles. An exemplary range to be administered is 10⁵ to 10¹⁰ virus particles per mammal, such as a human.

In some examples, a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or a nucleic acid encoding the mutated FGF1 protein, is administered in combination (such as sequentially or simultaneously or contemporaneously) with one or more other agents, such as those useful in the treatment of diabetes or insulin resistance (e.g., insulin).

Anti-diabetic agents are generally categorized into six classes: biguanides (e.g., metformin); thiazolidinediones (including rosiglitazone (Avandia®), pioglitazone (Actos®), rivoglitazone, and troglitazone); sulfonylureas; inhibitors of carbohydrate absorption; fatty acid oxidase inhibitors and anti-lipolytic drugs; and weight-loss agents. Any of these agents can also be used in the methods disclosed herein. The anti-diabetic agents include those agents disclosed in Diabetes Care, 22(4):623-634. One class of anti-diabetic agents of use is the sulfonylureas, which are believed to increase secretion of insulin, decrease hepatic glucogenesis, and increase insulin receptor sensitivity. Another class of anti-diabetic agents is the biguanide antihyperglycemics, which decrease hepatic glucose production and intestinal absorption, and increase peripheral glucose uptake and utilization, without inducing hyperinsulinemia.

In some examples, a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) can be administered in combination with effective doses of anti-diabetic agents (such as biguanides, thiazolidinediones, or incretins) and/or lipid lowering compounds (such as statins or fibrates). The terms “administration in combination,” “co-administration,” or the like, refer to both concurrent and sequential administration of the active agents. Administration of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25) or a nucleic acid encoding such a mutant FGF1 protein, may also be in combination with lifestyle modifications, such as increased physical activity, low fat diet, low sugar diet, and smoking cessation.

Additional agents that can be used in combination with the disclosed mutated FGF1 proteins include, without limitation, anti-apoptotic substances such as the Nemo-Binding Domain and compounds that induce proliferation such as cyclin dependent kinase (CDK)-6, CDK-4 and Cyclin D1. Other active agents can be utilized, such as antidiabetic agents for example, insulin, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570) and antagonists, PPAR-gamma/alpha modulators (such as KRP 297), alpha-glucosidase inhibitors (e.g., acarbose, voglibose), Dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g., exendin-4) or amylin. In some embodiments the agent is an immunomodulatory factor such as anti-CD3 mAb, growth factors such as HGF, vascular endothelial growth factor (VEGF), platelet derived growth factor (PDGF), lactogens, or parathyroid hormone related protein (PTHrP). In one example, the mutated FGF1 protein is administered in combination with a therapeutically effective amount of another FGF, such as FGF21, FGF19, or both, heparin, or combinations thereof.

In some embodiments, methods are provided for treating diabetes or pre-diabetes in a subject by administering a therapeutically effective amount of a composition including or a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or a nucleic acid encoding the mutated FGF1 protein, to the subject. The subject can have diabetes type I or diabetes type II. The subject can be any mammalian subject, including human subjects and veterinary subjects such as cats and dogs. The subject can be a child or an adult. The subject can also be administered insulin. The method can include measuring blood glucose levels.

In some examples, the method includes selecting a subject with diabetes, such as type I or type II diabetes, or a subject at risk for diabetes, such as a subject with pre-diabetes. These subjects can be selected for treatment with the disclosed mutated FGF1 proteins (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99% or 100% sequence identity to any one of SEQ ID NOS: 10-25) or nucleic acid molecules encoding such.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dL (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes

Care 33:S11-61, 2010).

In some examples, the subject treated with the disclosed compositions and methods has HbA1C of greater than 6.5% or greater than 7%.

In some examples, treating diabetes includes one or more of increasing glucose tolerance (such as an increase of at least 5%, at least 10%, at least 20%, or at least 50%, for example relative to no administration of the mutant FGF1, decreasing insulin resistance (for example, decreasing plasma glucose levels, decreasing plasma insulin levels, or a combination thereof, such as decreases of at least 5%, at least 10%, at least 20%, or at least 50%, for example relative to no administration of the mutant FGF1), decreasing serum triglycerides (such as a decrease of at least 10%, at least 20%, or at least 50%, for example relative to no administration of the mutant FGF1, decreasing free fatty acid levels (such as a decrease of at least 5%, at least 10%, at least 20%, or at least 50%, for example relative to no administration of the mutant FGF1), and decreasing HbA1c levels in the subject (such as a decrease of at least 0.5%, at least 1%, at least 1.5%, at least 2%, or at least 5% for example relative to no administration of the mutant FGF1). In some embodiments, the disclosed methods include measuring glucose tolerance, insulin resistance, plasma glucose levels, plasma insulin levels, serum triglycerides, free fatty acids, and/or HbA1c levels in a subject.

In some examples, administration of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or nucleic acid molecule encoding such, treats a metabolic disease, such as diabetes (such as type II diabetes) or pre-diabetes, by decreasing of HbA1C, such as a reduction of at least 0.5%, at least 1%, or at least 1.5%, such as a decrease of 0.5% to 0.8%, 0.5% to 1%, 1 to 1.5% or 0.5% to 2%. In some examples the target for HbA1C is less than about 6.5%, such as about 4-6%, 4-6.4%, or 4-6.2%. In some examples, such target levels are achieved within about 26 weeks, within about 40 weeks, or within about 52 weeks. Methods of measuring HbA1C are routine, and the disclosure is not limited to particular methods. Exemplary methods include HPLC, immunoassays, and boronate affinity chromatography.

In some examples, administration of a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or nucleic acid molecule encoding such, treats diabetes or pre-diabetes by increasing glucose tolerance, for example, by decreasing blood glucose levels (such as two-hour plasma glucose in an OGTT or FPG) in a subject. In some examples, the method includes decreasing blood glucose by at least 5% (such as at least 10%, at least 15%, at least 20%, at least 25%, at least 30%, at least 35%, or more) as compared with a control (such as no administration of any of insulin, a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or a nucleic acid molecule encoding such). In particular examples, a decrease in blood glucose level is determined relative to the starting blood glucose level of the subject (for example, prior to treatment with a mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or nucleic acid molecule encoding such). In other examples, decreasing blood glucose levels of a subject includes reduction of blood glucose from a starting point (for example greater than about 126 mg/dL FPG or greater than about 200 mg/dL OGTT two-hour plasma glucose) to a target level (for example, FPG of less than 126 mg/dL or OGTT two-hour plasma glucose of less than 200 mg/dL). In some examples, a target FPG may be less than 100 mg/dL. In other examples, a target OGTT two-hour plasma glucose may be less than 140 mg/dL. Methods to measure blood glucose levels in a subject (for example, in a blood sample from a subject) are routine.

In other embodiments, the disclosed methods include comparing one or more indicators of diabetes (such as glucose tolerance, triglyceride levels, free fatty acid levels, or HbA1c levels) to a control (such as no administration of any of insulin, any mutated FGF1 protein (such as one encoding a protein generated using the mutations shown in Table 1, the sequences in any one of SEQ ID NOS: 10-25, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-25), or a nucleic acid molecule encoding such), wherein an increase or decrease in the particular indicator relative to the control (as discussed above) indicates effective treatment of diabetes. The control can be any suitable control against which to compare the indicator of diabetes in a subject. In some embodiments, the control is a sample obtained from a healthy subject (such as a subject without diabetes). In some embodiments, the control is a historical control or standard reference value or range of values (such as a previously tested control sample, such as a group of subjects with diabetes, or group of samples from subjects that do not have diabetes). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study. In further examples, the control is the subject (or group of subjects) prior to treatment.

The disclosure is illustrated by the following non-limiting Examples.

### Example 1

### Preparation of Mutated FGF1 Proteins

Mutated FGF1 proteins can be made using known methods (e.g., see Xia et al., PLoS One. 7(11):e48210, 2012). An example is provided below.

Briefly, a nucleic acid sequence encoding an FGF1 mutant protein (e.g., any of SEQ ID NOS: 10-25) can be fused downstream of an enterokinase (EK) recognition sequence (Asp₄Lys) preceded by a flexible 20 amino acid linker (derived from the S-tag sequence of pBAC-3) and an N-terminal (His)₆ tag. The resulting expressed fusion protein utilizes the (His)₆ tag for efficient purification and can be subsequently processed by EK digestion to yield the mutant FGF1 protein.

The mutant FGF1 protein can be expressed from an E. coli host after induction with isopropyl-β-D-thio-galactoside. The expressed protein can be purified utilizing sequential column chromatography on Ni-nitrilotriacetic acid (NTA) affinity resin followed by ToyoPearl HW-405 size exclusion chromatography. The purified protein can be digested with EK to remove the N-terminal (His)₆ tag, 20 amino acid linker, and (Asp₄Lys) EK recognition sequence. A subsequent second Ni-NTA chromatographic step can be utilized to remove the released N-terminal mutant FGF1 protein (along with any uncleaved fusion protein). Final purification can be performed using HiLoad Superdex 75 size exclusion chromatography equilibrated to 50 mM Na₂PO₄, 100 mM NaCl, 10 mM (NH₄)₂SO₄, 0.1 mM ethylenediaminetetraacetic acid (EDTA), 5 mM L-Methionine, pH at 6.5 (“PBX” buffer); L-Methionine can be included in PBX buffer to limit oxidization of reactive thiols and other potential oxidative degradation.

In some examples, the enterokinase is not used, and instead, an FGF1 mutant protein (such as one that includes an N-terminal methionine) can be made and purified using heparin affinity chromatography.

For storage and use, the purified mutant FGF1 protein can be sterilely filtered through a 0.22 micron filter, purged with N2, snap frozen in dry ice and stored at −80° C. prior to use. The purity of the mutant FGF1 protein can be assessed by both Coomassie Brilliant Blue and Silver Stain Plus (BIO-RAD Laboratories, Inc., Hercules Calif.) stained sodium dodecyl sulfate polyacrylamide gel electrophoresis (SDS PAGE). Mutant FGF1 proteins can be prepared in the absence of heparin. Prior to IV bolus, heparin, or PBS, can be added to the protein.

In some examples, a mutant FGF1 protein (e.g., any one of SEQ ID NOS: 10-25) can be expressed in Escherichia coli cells and purified from the soluble bacterial cell lysate fraction by heparin affinity, ion exchange, and size exclusion chromatographies.

### Example 2

### Testing of FGF1 Mutants for Glucose Lowering Ability and Mitogenic Activity

This example describes how FGF1 mutant proteins provided herein (e.g., any of SEQ ID NOS: 10-25, or variants thereof) are tested, for example for their ability to lower blood glucose or treat a metabolic disease in vivo. In vitro mitogenic assays are also described. Similar methods can be used to test other FGF1 mutant proteins.

### Animals

Mice are housed in a temperature-controlled environment with a 12-hour light/12-hour dark cycle and handled according to institutional guidelines complying with U.S. legislation. Male ob/ob mice (B6.V-Lep^{ob}/J, Jackson laboratories) and male C57BL/6J mice receive a standard or high fat diet (MI laboratory rodent diet 5001, Harlan Teklad; high fat (60%) diet F3282, Bio-Serv) and acidified water ad libitum. STZ-induced diabetic mice on the C57BL/6J background can be purchased from Jackson laboratories. Mice are injected subcutaneously with 0.1 to 1 mg/ml (such as 0.5 mg/ml) solutions in PBS of the FGF1 mutant or PBS alone.

### Serum Analysis

Blood is collected by tail bleeding either in the ad libitum fed state or following overnight fasting. Free fatty acids (Wako), triglycerides (Thermo) and cholesterol (Thermo) can be measured using enzymatic colorimetric methods following the manufacturer's instructions. Serum insulin levels are measured using an Ultra-Sensitive Insulin ELISA kit (Crystal Chem). Plasma adipokine and cytokine levels can be measured using Milliplex™ MAP and Bio-Plex Pro™ kits (Millipore and Bio-Rad).

### Metabolic Studies

Glucose tolerance tests (GTT) are conducted after o/n fasting. Mice are injected i.p. with 1 g of glucose per/kg bodyweight and blood glucose is monitored at 0, 15, 30, 60, and 120 min using a OneTouch Ultra glucometer (Lifescan Inc). Insulin tolerance tests (ITT) are conducted after 3 h fasting. Mice are injected i.p. with 2U of insulin/kg bodyweight (Humulin R; Eli Lilly) and blood glucose is monitored at 0, 15, 30, 60, and 90 min using a OneTouch Ultra glucometer (Lifescan Inc). Real-time metabolic analyses can be conducted in a Comprehensive Lab Animal Monitoring System (Columbus Instruments). CO₂ production, O₂ consumption, RQ (relative rates of carbohydrate versus fat oxidation), and ambulatory counts are determined for six consecutive days and nights, with at least 24 h for adaptation before data recording. Total body composition analysis is performed using an EchoMRI-100™ (Echo Medical Systems, LLC)

### Mitogenic Assay

Low passage NIH-3T3 cells are cultured in 10% FBS DMEM high glucose until 70-80% confluence. On day 1, cells are trypsinized and plated in white wall 96-well plate at 5000 cells/well in 10% FBS-DMEM high glucose medium (100 μl per well). 24 hours later, cells are washed in PBS and the medium is replaced with proliferation medium (DMEM high glucose without FBS, 25 μg/ml sodium heparin) and various concentrations of human recombinant FGF1 (R&D Systems) (0, 0.00001, 0.0001, 0.001, 0.002, 0.005, 0.01, 0.1, 0.5, 1, 10, 50 ng/ml, final concentration in 100 μl total medium) or with the same amount of an FGF1 mutant protein. Cells are allowed to proliferate for 24 hours. Cellular proliferation is measured by direct addition of 50 μl of CellTiter Glo reagent into 100 μl of medium. Luminance is quantified after 10-minute incubation at room temperature. The luminance is plotted against log 2 transformed concentration and fitted with 3-parameter curve fitting algorithm using Graphpad Prism.

### Results

A mutant FGF1 protein was generated that contained an internal non-native disulfide bond between amino acids 66 and 83 (SEQ ID NO: 10), to increase stability of the protein and reduce its mitogenicity. FIG. 3A shows the blood glucose lowering ability of SEQ ID NO: 10, as compared to native FGF1 (SEQ ID NO: 5). At 24 hours, SEQ ID NO: 10 lowered blood glucose to a greater extent than FGF1. In addition, SEQ ID NO: 10 had less mitogenic activity than FGF1 (the EC₅₀ for mitogenicity was shifted by several orders of magnitude) (FIG. 3B). Therefore, the presence of an internal non-native disulfide bond can be used to lower the mitogenic activity of FGF1, without adverse effects on desired glucose lowering activity.

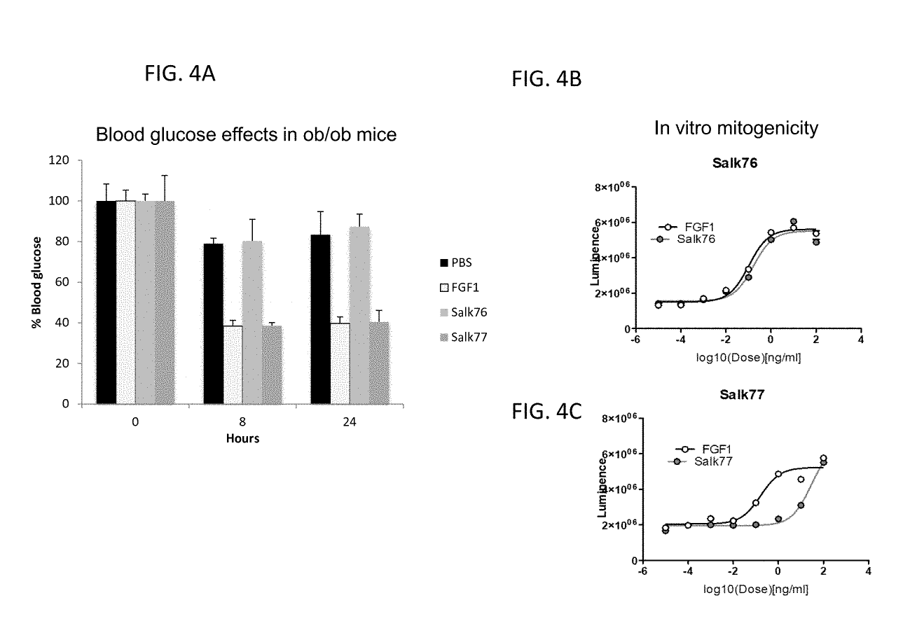

Other mutant FGF1 proteins were generated that contained point mutations to explore receptor interactions (K12, Y55, E87, N95) in combination with mutations to improve pharmacokinetic stability (C117) (SEQ ID NOS: 11 and 12), to increase stability of the protein and reduce mitogenicity. FIG. 4A shows the blood glucose lowering ability of SEQ ID NOS: 11 and 12, as compared to native FGF1 (SEQ ID NO: 5) and PBS. At 8 and 24 hours, SEQ ID NO: 12, but not SEQ ID NO: 11, was able to lower blood glucose as well as native FGF1. In addition, SEQ ID NO: 12, but not SEQ ID NO: 11 had less mitogenic activity than FGF1 (FIGS. 4B and 4C). Therefore, SEQ ID NO: 12 demonstrates that the presence of point mutations in the loop region between beta sheets 9 and 10 can be used to lower the mitogenic activity of FGF1, without adverse effects on desired glucose lowering activity.

Mutant FGF1 proteins were generated that contained deletion of 9 (FGF1 (10-140 αα)) or 13 (FGF1 (14-140 αα)) consecutive N-terminal amino acids (SEQ ID NOS: 16 and 17), to reduce its mitogenicity and point mutations to increase thermostability. FIG. 5A shows the blood glucose lowering ability of SEQ ID NOS: 16 and 17, as compared to native FGF1 (SEQ ID NO: 5) and PBS. Both SEQ ID NO: 16 and 17 had weak effects on glucose lowering, indicating that deletion of 9 or 13 N-terminal amino acids can have undesirable effects on blood glucose lowering activity. However, both SEQ ID NOS: 16 and 17 had significantly less mitogenic activity than FGF1 (FIGS. 5B and 5C), with SEQ ID NO: 17 having no detectable mitogenicity.

Based on these results, additional FGF1 mutant proteins having the N-terminal deleted amino acids replaced with the engineered MRDSSPL sequence (see SEQ ID NOS: 13-15 and 20-21) or containing additional point mutations (see SEQ ID NOS: 18-19 and 22-25), can be tested for their ability to significantly lower glucose with and reduce mitogenicity, as compared to native FGF1.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. An isolated mutated mature fibroblast growth factor (FGF) 1 protein comprising at least 80%, at least 85%, at least 90%, at least 95%, at least 96%, at least 97%, at least 99%, or 100% sequence identity to any one of SEQ ID NOS: 10, 11, 12, 13, 14, 15, 16, 17, 18, 19, 20, 21, 22, 23, 24 or 25.

2. The isolated mutated mature FGF1 protein of claim 1, wherein the mutated mature FGF1 protein comprises a deletion of at least 9, at least 10, at least 11, at least 12, or at least 13 contiguous N-terminal amino acids from the native FGF1 protein, wherein the mutated FGF1 protein has reduced mitogenic activity as compared to a wild-type mature FGF1 protein.

3. The isolated mutated mature FGF1 protein of claim 1, wherein the mutated mature FGF1 protein comprises at least one point mutation shown in Table 1.

4. The isolated mutated mature FGF1 protein of claim 1, wherein the mutated mature FGF1 protein comprises one or more point mutations selected from the group consisting of: K12V, H21Y, Q40K, L44F, S47A, S47V, S47I, Y55F, Y55V, Y55S, Y55A, Y55W, A66C, C83T, C83S, C83A, C83V, E87Q, E87D, E87V, E87A, E87S, E87T, E87H, H93G, H93A, N95V, N95A, N95S, N95T, S99A, K101E, H102Y, H102A, W107A F108Y, S116R, C117V, C117P, C117T, C117S, C117A, and F132W wherein the numbering refers to the sequence shown SEQ ID NO: 5.

5. The isolated mutated mature FGF1 protein of claim 1, wherein the mutated mature FGF1 protein comprises mutations at one or more of S99, K101, H102, and W107, wherein the numbering refers to SEQ ID NO: 5.

6. The isolated mutated mature FGF1 protein of claim 1, wherein the mutated mature FGF1 protein comprises a H21Y, L44F, H102Y, and/or F¹⁰⁸Y mutation.

7. The isolated mutated mature FGF1 protein of claim 1, wherein a wild-type mature FGF1 protein comprises SEQ ID NO: 5.

8. The isolated mutated mature FGF1 protein of claim 1, wherein the mutated mature FGF1 protein comprises the protein sequence of SEQ ID NO: 10, 11, 12, 13, 14, 15, 16, 17, 18, 19, 20, 21, 22, 23, 24 or 25.

9. The isolated mutated mature FGF1 protein of claim 1, wherein the N-terminal amino acid is a methionine.

10. The isolated mutated mature FGF1 protein of claim 1, wherein the protein has decreased mitogenicity compared to a native mature FGF1 protein.

11. The isolated mutated mature FGF1 protein of claim 1, wherein the protein has increased blood glucose lowering ability compared to a native mature FGF1 protein.

12. An isolated nucleic acid molecule encoding the mutated mature FGF1 protein of claim 1.

13. A nucleic acid vector comprising the isolated nucleic acid molecule of claim 12.

14. A host cell comprising the nucleic acid vector of claim 13.

15. A method of reducing blood glucose in a mammal, comprising:
administering to the mammal a therapeutically effective amount of the mutated mature FGF1 protein of claim 1, an isolated nucleic acid molecule encoding the mutated FGF1 protein of claim 1, or a nucleic acid vector of comprising the isolated nucleic acid molecule encoding the mutated FGF1 protein of claim 1, thereby reducing blood glucose in the mammal.

16. A method of treating a metabolic disease in a mammal, comprising:
administering to the mammal a therapeutically effective amount of the mutated mature FGF1 protein of claim 1, an isolated nucleic acid molecule encoding the mutated FGF1 protein of claim 1, or a nucleic acid vector comprising the isolated nucleic acid molecule encoding the mutated FGF1 protein of claim 1, thereby treating the metabolic disease.

17. The method of claim 16, wherein the metabolic disease is type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), hyperlipidemia, hypertension, latent autoimmune diabetes (LAD), or maturity onset diabetes of the young (MODY).

18. A method of reducing fed and fasting blood glucose, improving insulin sensitivity and glucose tolerance, reducing systemic chronic inflammation, ameliorating hepatic steatosis in a mammal, reducing food intake, or combinations thereof, comprising:
administering to the mammal a therapeutically effective amount of the mutated mature FGF1 protein of claim 1, an isolated nucleic acid molecule encoding the mutated FGF1 protein of claim 1, or a nucleic acid vector comprising the isolated nucleic molecule encoding the mutated FGF1 protein of claim 1, thereby reducing fed and fasting blood glucose, improving insulin sensitivity and glucose tolerance, reducing systemic chronic inflammation, ameliorating hepatic steatosis in a mammal, reducing food intake, or combinations thereof.

19. The method of claim 15, wherein the therapeutically effective amount of the mutated mature FGF1 protein is at least 0.1 mg/kg.

20. The method of claim 15, wherein the administering is subcutaneous, intraperitoneal, intramuscular, intravenous or intrathecal.

21. The method of claim 15, wherein the mammal is a cat or dog.

22. The method of claim 15, wherein the mammal is a human.

23. The method of claim 15, wherein the method further comprises administering an additional therapeutic compound.

24. The method of claim 23, wherein the additional therapeutic compound is insulin, an alpha-glucosidase inhibitor, amylin agonist, dipeptidyl-peptidase 4 (DPP-4) inhibitor, meglitinide, sulfonylurea, a peroxisome proliferator-activated receptor (PPAR)-gamma agonist, or combinations thereof.

25. The method of claim 24, wherein the PPAR-gamma agonist is a thiazolidinedione (TZD), aleglitazar, farglitazar, muraglitazar, or tesaglitazar.

26. The method of claim 25, wherein the TZD is pioglitazone, rosiglitazone, rivoglitazone, or troglitazone.


SEQUENCE LISTING (26 sequences)

SEQ ID NO: 1 - DNA, 468 bp, Homo sapiens
  CDS (1)..(468)
atggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaa

SEQ ID NO: 2 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - DNA, 468 bp, Mus musculus
  CDS (1)..(468)
atggctgaaggggagatcacaaccttcgcagccctgaccgagaggttcaacctgcctctaggaaactacaaaaagcccaaactgctctactgcagcaacgggggccacttcttgaggatccttcctgatggcaccgtggatgggacaagggacaggagcgaccagcacattcagctgcagctcagtgcggaaagtgcgggcgaagtgtatataaagggtacggagaccggccagtacttggccatggacaccgaagggcttttatacggctcgcagacaccaaatgaggaatgtctgttcctggaaaggctggaagaaaaccattataacacttacacctccaagaagcatgcggagaagaactggtttgtgggcctcaagaagaacgggagctgtaagcgcggtcctcggactcactatggccagaaagccatcttgtttctgcccctcccggtgtcttctgactag

SEQ ID NO: 4 - protein, 155 aa, Mus musculus
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 5 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 6 - protein, 155 aa, Gorilla gorilla; identical to SEQ ID NO: 2

SEQ ID NO: 7 - protein, 155 aa, Pan troglodytes; identical to SEQ ID NO: 2

SEQ ID NO: 8 - protein, 155 aa, Canis familiaris
MAEGEITTFTALTEKFNLPPGNYMKPKLLYCSNGGHFLRILPDGTVDGTRDRSDQHIQLQLSAESVGEVYIKSTETGQYLAMDTDGLLYGSQTPNEECLFLERLEENHYNTYTSKKHAEKNWFVGLKKNGSCKRGPRTHYGQKAILFLPLPVSSD

SEQ ID NO: 9 - protein, 155 aa, Felis catus
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYTSKKHAEKNWFVGLKKNGSCKRGPRTHYGQKAILFLPLPVSSD

SEQ ID NO: 10 - protein, 140 aa, Artificial Sequence
  mutant FGF1
FNLPPGNYKKPVLLYCSNGGHFLRILPDGTVDGTRDRSDQHIQLQLSAESVGEVYIKSTETGQYLCMDTDGLLYGSQTPNEECLFLERLEENHYVTYISKKHAEKNWFVGLKKNGSVKRGPRTHYGQKAILFLPLPVSSD

SEQ ID NO: 11 - protein, 140 aa, Artificial Sequence
  mutant FGF1
FNLPPGNYKKPKLLYCSNGGHFLRILPDGTVDGTRDRSDQHIQLQLSAESVGEVWIKSTETGQYLAMDTDGLLYGSQTPNEECLFLHRLEENHYNTYISKKHAEKNWFVGLKKNGRVKRGPRTHYGQKAILFLPLPVSSD

SEQ ID NO: 12 - protein, 140 aa, Artificial Sequence
  mutant FGF1
FNLPPGNYKKPVLLYCSNGGHFLRILPDGTVDGTRDRSDQHIQLQLSAESVGEVWIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 13 - protein, 137 aa, Artificial Sequence
  mutant FGF1
MRDSSPLPVLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 14 - protein, 137 aa, Artificial Sequence
  mutant FGF1
MRDSSPLPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 15 - protein, 137 aa, Artificial Sequence
  mutant FGF1
MRDSSPLPVLLYCSNGGHFLRILPDGTVDGTRDRSDQHIQLQLSAESVGEVYIKSTETGQYLAMDTDGLLYGSQTPNEECLFLVRLEENHYNTYISKKHAEKNWFVGLKKNGSVKRGPRTHYGQKAILFLPLPVSSD

SEQ ID NO: 16 - protein, 127 aa, Artificial Sequence
  mutant FGF1
LYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 17 - protein, 127 aa, Artificial Sequence
  mutant FGF1
LYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSCKRGPRTHYGQKAILFLPLPVSSD

SEQ ID NO: 18 - protein, 127 aa, Artificial Sequence
  mutant FGF1
LYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 19 - protein, 127 aa, Artificial Sequence
  mutant FGF1
LYCSNGGHFLRILPDGTVDGTRDRSDQHIQFQLSAESVGEVYIKSTETGQYLCMDTDGLLYGSQTPNEETLFLERLEENHYVTYISKKHAEKNWFVGLKKNGSVKRGPRTHYGQKAILWLPLPVSSD

SEQ ID NO: 20 - protein, 134 aa, Artificial Sequence
  mutant FGF1
MRDSSPLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 21 - protein, 138 aa, Artificial Sequence
  mutant FGF1
MRDSSPLGGQVLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 22 - protein, 129 aa, Artificial Sequence
  mutant FGF1
VLLYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 23 - protein, 129 aa, Artificial Sequence
  mutant FGF1
VLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSCKRGPRTHYGQKAILFLPLPVSSD

SEQ ID NO: 24 - protein, 129 aa, Artificial Sequence
  mutant FGF1
VLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 25 - protein, 140 aa, Artificial Sequence
  mutant FGF1
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYIAKEAAEKNWFVGLKKNGSCKRGPRTHYGQKAILFLPLPVSSD

SEQ ID NO: 26 - protein, 142 aa, Artificial Sequence
  portion of FGF2
PALPEDGGAAFPPGHFKDPKRLYCKNGGFFLRIHPDGRVREKSDPHVKLQLQAEERGVVSIKGVCANRYLAMKEDGRLLASKCVTEECFFFERLESNNYNTYRSRKYSSWYVALKRTGQYKLGSKTGPGQKAILFLFMSAKS